(19) United States
(12) Patent Application Publication
(10) Pub. No.: US 2021/0332088 A1
(43) Pub. Date: Oct. 28, 2021

(54) Multimerizing Polypeptides Derived From Jelly Roll Fold Domain of Adenovirus Penton Base

(71) Applicant: Imophoron Limited, Bristol (GB)

(72) Inventor: Frédéric Garzoni, Bristol (GB)

(21) Appl. No.: 17/264,569

(22) PCT Filed: Jul. 31, 2019
(86) PCT No.: PCT/EP2019/070722

§ 371 Date: Jan. 29, 2021

(30) Foreign Application Priority Data
Jul. 31, 2018 (EP) 18186731.8

(51) Int. Cl. C07K 14/005; C12N 7/00

(52) U.S. Cl. CPC C07K 14/005; C12N 7/00; C12N 2710/10322; C12N 2770/36134; C12N 2710/10334; C12N 2770/36122; C12N 2710/10323

(57) ABSTRACT

The present invention relates to novel polypeptide scaffolds for optimized presentation of oligopeptides, polypeptide sequences, protein domains, proteins and protein complexes made up of two, several or many subunits. These oligopeptides, polypeptide sequences, protein domains and proteins presented by the polypeptide scaffolds of the invention can include antigenic entities that stimulate the immune system to trigger an immune response, for example for vaccination purposes, or for preparing antibodies or other binder molecules in cell culture, or in vitro in a test tube. In a preferred embodiment, the polypeptides of the invention are assembled into Virus Like Particles (VLPs) optimized for presentation of antigens useful in the context of vaccination against infectious agents or tumors.

### FIELD OF THE INVENTION

The present invention relates to the design and production of novel polypeptide scaffolds for optimized presentation of oligopeptides, polypeptide sequences, protein domains, proteins and/or protein complexes made up of two, several or many subunits. These oligopeptides, polypeptide sequences, protein domains and/or proteins presented by the polypeptide scaffolds of the invention can include antigenic entities that stimulate the immune system to trigger an immune response, for example for vaccination purposes, or for preparing antibodies or other binder molecules in cell culture, or in vivo, or in vitro in a test tube. In a preferred embodiment, the polypeptides of the invention are assembled into Virus Like Particles (VLPs) optimized for presentation of antigens useful in the context of vaccination against infectious agents or tumors.

### REFERENCE TO SEQUENCE LISTING

This application contains a Sequence Listing. The application thus incorporates by reference the material in the ASCII text file 07916_P0001A.txt, created on Jan. 27, 2021, and having a size of 108,656 bytes.

### BACKGROUND OF THE INVENTION

A prerequisite for successful protein scaffold design for presentation of oligopeptides, polypeptide sequences, protein domains, proteins and/or protein complexes, is a compact, stable multimerization domain which can accommodate modalities representing exposed and flexible loop structures that can accommodate such oligopeptides, polypeptide sequences, protein domains, proteins and/or protein complexes. Preferably, these displayed entities can represent immunogenic antigens that are presented to an immune system. Penton base proteins (protomers) from a number of Adenovirus (Ad) serotypes assemble into pentamers which then form dodecahedra, resembling virus-like particles. In contrast to live virus, they carry no genetic material such that these VLPs are beneficial under safety considerations.

Adenovirus is one of the most commonly used gene therapy vector in humans. The adenovirus shell is predominantly made up of two distinct proteins, the hexon protein, and the penton base protein, with the latter forming pentameric assemblies to which attach the fibres characteristic for this virus. Penton base proteins of certain adenovirus serotypes were shown to spontaneously self-assemble into a multimeric superstructure when expressed recombinantly in absence of other adenoviral components. This superstructure represents a dodecamer, formed by a total of 60 adenovirus base proteins arranged in twelve identical copies of a pentameric ‘crown-shaped’ assembly (FIG. 1). The adenovirus base protein itself adopts a two-domain architecture with one domain representing a beta-barrel conjoined to a second domain stabilized by alpha-helices (FIG. 1B). The former mediates multimerization into the dodecahedron as evidenced by mutational studies, while the latter presents extended loops to the solvent on the dodecahedron surface. These loops are extremely variable in length and sequence content in different adenovirus serotypes, while the remainder of the base protein is highly conserved throughout the species. The adenovirus dodecahedron represents a highly versatile display scaffold for example for immunogenic peptides that can be inserted into the loops replacing naturally occurring sequences. Literally hundreds of heterologous peptides can thus be displayed efficiently on a single dodecahedron, if all insertion sites are occupied. The dodecahedron can be produced recombinantly in very high amount, it is exceptionally stable and can be stored at ambient temperature for indefinite time. Exploiting these highly advantageous characteristics, synthetic dodecahedron-based particles displaying immunogenic peptides in their exposed loops have been engineered for potential use in a range of applications including onco-immunology and emergent infectious disease.

WO2017167988 A1 describes synthetic adenovirus dodecahedrons facilitating epitope insertion into the exposed loops and also discloses an adenovirus base protein production protocol.

The problem underlying the present invention is the provision of a novel system for presenting antigens or other cargo through protein scaffolds which can assemble into VLP structures.

The above technical problem is provided by the embodiments of the present invention as defined in the claims as well as further described herein and illustrated by the accompanying drawings.

### SUMMARY OF THE INVENTION

The present invention is based, at least in part, on the finding that the architecture of the adenovirus penton base proteins represents a bona fide two-domain structure which may have arisen during evolution by gene fusion (FIG. 1B). The two domains as it appeared could be easily split into two distinct compact entities: the beta-barrel containing the multimerization information, and the alpha-helical domain resembling a “crown”.

Therefore, according to the present invention, there is provided a “minimal” multimerization polypeptide which can be coupled to antigen or other cargo carrying entities which utmost versatility and flexibility. The thus engineered polypeptide of the invention is derived from the amino acid sequences of adenovirus penton bases (also referred to herein as “penton base protomers”) which form the beta-barrel domain of the adenovirus penton base. The beta-barrel domain of adenovirus penton base proteins forms a so-called jellyroll fold domain comprising eight beta-sheets 1 through 8 (see FIG. 2); cf. Zubieta et al. (2005) Mol. Cell 17, 121-135. According to the present invention, it has been surprisingly found that, for effective multimerization and thus, display of coupled cargo such as oligopeptides or polypeptides like antigens or other coupled entities, e.g. drugs, labels, nucleic acids, the two loops (forming the “crown” domain) interspersed in the sequence between the amino acid stretches forming the jellyroll fold domain, can be completely, or in other embodiments partially, replaced by desired non-adenoviral sequences such as oligopeptide linkers (to which antigens or other cargo can in turn be coupled) or any desired amino acid sequence such as polypeptides, proteins, protein domains, protein complexes etc.

Therefore, in preferred embodiments of the invention, a nucleic acid, a drug, label and/or binding partner of a biological binding pair is/are coupled to L₁ and/or L₂. “Biological binding” pair according to the invention are pairs of biological entities or compounds, respectively, which are typically found in nature or which are at least derived from binding pairs found in nature. Examples include, but are not limited to, antigens, antibodies, antibody fragments, diabodies, antibody mimetics, receptors and their ligands, biotin, streptavidin and the like.

Such entities may be coupled to L₁ and/or L₂ via means known in the art. If necessary linkers of any type can be linked to a suitable group at a position in L₁ and/or L₂, which linker is then coupled to the desired entity. Typical groups present in L₁ and/or L₂ which can be engaged into a chemical coupling include NH₂ and SH groups of amino acid residues present in L₁ and/or L₂. However, the coupling of cargo to L₁ and/or L₂ is not restricted to chemical bonds but also include any other interaction such as ionic interactions, hydrogen bonds and Van der Waals interactions.

The jellyroll fold domain according to the invention is formed by three amino acid stretches (which also may be referred to as, e.g. “segments” or “regions”): an N-terminal stretch, an intermediate stretch, and a C-terminal stretch. In the native adenovirus penton base protomer, the loop segments are found between the N-terminal amino acid stretch and the intermediate stretch (large loop) and between the intermediate amino acid stretch and the C-terminal amino acid stretch (small loop). As outlined above, the typically non-adenoviral sequences of the polypeptide of the present invention, which may be denoted herein as “linkers”, replace the loop segments of the native adenovirus penton base protomer. In other embodiments of the invention, one of the large loops and the small loop of the native penton base may be present in the polypeptide of the invention and forming L₁ or L₂.

Therefore, the polypeptide according to the invention generally has a structure represented by the following general formula (I).

A-L₁-B-L₂-C (I)
wherein
A is an N-terminal amino acid stretch of an adenovirus penton base protein;
B is an amino acid stretch of an adenovirus penton base protein;
C is a C-terminal amino acid stretch of an adenovirus penton base;
wherein B is an amino acid stretch located between A and C in the sequence of said adenovirus penton base;
wherein A, B and C form the jellyroll fold domain of said adenovirus penton base protein.

L₁ and L₂ are the linkers as outlined above. Thus, L₁ and L₂ can be selected from almost any amino acid sequence (as long as the same does not interfere with the multimerization of the polypeptide). Thus. L₁ and L₂ may be the same or different and are independently from one another selected from the group consisting of an oligopeptide, a polypeptide, a protein and a protein complex. The sequences of L₁ and L₂ are typically non-adenoviral, i.e. have an amino acid sequence of at least 5, 6, 7, 8, 9 10 or more amino acids, which sequence does not exist or occur in the known penton base protomer sequences of any adenovirus serotype, more preferably in any adenoviral protein.

In an alternative embodiment of the invention, the linkers L₁ and L₂ may be selected from the loop sequences (i.e. regions comprising the first and second RGD loops and/or the variable loop as defined in WO 2017/167988 A1) of a penton base of an adenovirus. However, in this embodiment, the sequences of the loop segments are derived from an adenovirus having a different serotype compared to the serotype of the adenovirus from which said amino acid stretches A. B and C are derived. Accordingly, this embodiment of the invention provides chimeras of penton base protomers where the beta-barrel, jellyroll fold domain is derived from one adenovirus subtype, whereas L₁ and L₂ are polypeptides comprising RGD loop segments and/or VL variable loop segments (forming the “crown” domain) are derived from an adenovirus subtype different from the adenovirus subtype the jellyroll fold domain is derived from.

In a preferred embodiment of the invention, referring to FIG. 2, amino acid stretch A comprises beta-sheets 1, 2 and 3 of the jellyroll fold domain of the adenovirus penton base protomer, amino acid stretch B comprises beta sheets 4 and 5 of the jellyroll fold domain of said adenovirus penton base protomer, and amino acid stretch C comprises beta sheets 6, 7 and 8 of the jellyroll fold domain of said adenovirus penton base protomer. It is to be understood that each segment A, B, and C can be independently derived from the same or different adenoviruses.

Preferably, amino acid stretches A, B, and C have an amino acid sequence which is each independently derived from penton base sequences selected from the group consisting of penton bases of human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53).

Preferred amino acid sequences of the above-indicated adenovirus penton bases are laid down in generally accessible databases such as UniProt and UniProtE, and especially preferred sequences referred to herein for the above-mentioned adenovirus subtypes are laid down in UniProt Acc. No. Q2Y0H9 (human adenovirus serotype 3; SEQ ID NO: 1), UniProtAcc. No. P03276 (human adenovirus serotype 2; SEQ ID NO: 2), UniProt Acc. No. Q2KSF3 (human adenovirus serotype 4; SEQ ID NO: 3), UniProtAcc. No. P12538 (human adenovirus serotype 5; SEQ ID NO: 4), UniProt Acc. No. Q9JFT6 (human adenovirus serotype 7; SEQ ID NO: 5), UniProt Acc. No. D2DM93 (human adenovirus serotype 11; SEQ ID NO: 6), UniProt Acc. No. P36716 (human adenovirus serotype 12; SEQ ID NO: 7), UniProt Acc. No. F1DT65 (human adenovirus serotype 17; SEQ ID NO: 8), UniProt Acc. No. M0QUK0 (human adenovirus serotype 25; SEQ ID NO: 9), UniProt Acc. No. Q7T941 (human adenovirus serotype 35; SEQ ID NO: 10), UniProtAcc. No. Q912J1 (human adenovirus serotype 37; SEQ ID NO: 11), UniProt Acc. No. F8WQN4 (human adenovirus serotype 41; SEQ ID NO: 12), UniProt Acc. No. E5L3Q9 (gorilla adenovirus; SEQ ID NO: 13), UniProt Acc. No. G9G849 (chimpanzee adenovirus; SEQ ID NO: 14), UniProt Acc. No. H8PFZ9 (simian adenovirus serotype 18; SEQ ID NO: 15), UniProt Acc. No. F6KSU4 (simian adenovirus serotype 20; SEQ ID NO: 16), UniProt Acc. No. F2WTK5 (simian adenovirus serotype 49; SEQ ID NO: 17), UniProt Acc. No. A0A0A1EWW1 (rhesus adenovirus serotype 51; SEQ ID NO: 18), UniProt Acc. No. A0A0A1EWX7 (rhesus adenovirus serotype 52; SEQ ID NO: 19), and UniProt Acc. No. A0A0A1 EWZ7 (rhesus adenovirus serotype 53; SEQ ID NO: 20).

The amino acid sequences of the above penton bases are as follows (the respective UniProt Acc. No. is indicated in brackets):

Human Adenvirus Serotype 3 poenton base hAd3
(Q2Y0H9); SEQ ID NO: 1:
MRRRAVLGGA VVYPEGPPPS YESVMQQQAA MIQPPLEAPF
VPPRYLAPTE GRNSIRYSEL SPLYDTTKLY LVDNKSADIA
SLNYQNDHSN FLTTVVQNND FTPTEASTQT INFDERSRWG
GQLKTIMHTN MPNVNEYMFS NKFKARVMVS RKAPEGVTVN
DTYDHKEDIL KYEWFEFILP EGNFSATMTI DLMNNAIIDN
YLEIGRQNGV LESDIGVKFD TRNFRLGWDP ETKLIMPGVY
TYEAFHPDIV LLPGCGVDFT ESRLSNLLGI RKRHPFQEGF
KIMYEDLEGG NIPALLDVTA YEESKKDTTT ETTTLAVAEE
TSEDDDITRG DTYITEKQKR EAAAAEVKKE LKIQPLEKDS
KSRSYNVLED KINTAYRSWY LSYNYGNPEK GIRSWTLLTT
SDVTCGAEQV YWSLPDMMQD PVTFRSTRQV NNYPVVGAEL
MPVFSKSFYN EQAVYSQQLR QATSLTHVFN RFPENQILIR
PPAPTITTVS ENVPALTDHG TLPLRSSIRG VQRVTVTDAR
RRTCPYVYKA LGIVAPRVLS SRTF
hAd2 (P03276); SEQ ID NO: 2:
MQRAAMYEEG PPPSYESVVS AAPVAAALGS PFDAPLDPPF
VPPRYLRPTG GRNSIRYSEL APLFDTTRVY LVDNKSTDVA
SLNYQNDHSN FLTTVIQNND YSPGEASTQT INLDDRSHWG
GDLKTILHTN MPNVNEFMFT NKFKARVMVS RSLTKDKQVE
LKYEWVEFTL PEGNYSETMT IDLMNNAIVE HYLKVGRQNG
VLESDIGVKF DTRNFRLGFD PVTGLVMPGV YTNEAFHPDI
ILLPGCGVDF THSRLSNLLG IRKRQPFQEG FRITYDDLEG
GNIPALLDVD AYQASLKDDT EQGGDGAGGG NNSGSGAEEN
SNAAAAAMQP VEDMNDHAIR GDTFATRAEE KRAEAEAAAE
AAAPAAQPEV EKPQKKPVIK PLTEDSKKRS YNLISNDSTF
TQYRSWYLAY NYGDPQTGIR SWTLLCTPDV TCGSEQVYWS
LPDMMQDPVT FRSTSQISNF PVVGAELLPV HSKSFYNDQA
VYSQLIRQFT SLTHVFNRFP ENQILARPPA PTITTVSENV
PALTDHGTLP LRNSIGGVQR VTITDARRRT CPYVYKALGI
VSPRVLSSRT F
hAd4 (Q2KSF3); SEQ ID NO: 3:
MMRRAYPEGP PPSYESVMQQ AMAAAAAIQP PLEAPYVPPR
YLAPTEGRNS IRYSELTPLY DTTRLYLVDN KSADIASLNY
QNDHSNFLTT VVQNNDFTPT EASTQTINFD ERSRWGGQLK
TIMHTNMPNV NQFMYSNKFK ARVMVSRKTP NGVTVGDNYD
GSQDELKYEW VEFELPEGNF SVTMTIDLMN NAIIDNYLAV
GRQNGVLESD IGVKFDTRNF RLGWDPVTEL VMPGVYTNEA
FHPDIVLLPG CGVDFTESRL SNLLGIRKRQ PFQEGFQIMY
EDLDGGNIPA LLDVEAYEKS KEESVAAATT AVATASTEVR
DDNFASAAAV AAVKADETKS KIVIQPVEKD SKERSYNVLS
DKKNTAYRSW YLAYNYGDRD KGVRSWTLLT TSDVTCGVEQ
VYWSLPDMMQ DPVTFRSTHQ VSNYPVVGAE LLPVYSKSFF
NEQAVYSQQL RAFTSLTHVF NRFPENQILV RPPAPTITTV
SENVPALTDH GTLPLRSSIR GVQRVTVTDA RRRTCPYVYK
ALGIVAPRVL SSRTF
hAd5 (P12538); SEQ ID NO: 4:
MRRAAMYEEG PPPSYESVVS AAPVAAALGS PFDAPLDPPF
VPPRYLRPTG GRNSIRYSEL APLFDTTRVY LVDNKSTDVA
SLNYQNDHSN FLTTVIQNND YSPGEASTQT INLDDRSHWG
GDLKTILHTN MPNVNEFMFT NKFKARVMVS RLPTKDNQVE
LKYEWVEFTL PEGNYSETMT IDLMNNAIVE HYLKVGRQNG
VLESDIGVKF DTRNFRLGFD PVTGLVMPGV YTNEAFHPDI
ILLPGCGVDF THSRLSNLLG IRKRQPFQEG FRITYDDLEG
GNIPALLDVD AYQASLKDDT EQGGGGAGGS NSSGSGAEEN
SNAAAAAMQP VEDMNDHAIR GDTFATRAEE KRAEAEAAAE
AAAPAAQPEV EKPQKKPVIK PLTEDSKKRS YNLISNDSTF
TQYRSWYLAY NYGDPQTGIR SWTLLCTPDV TCGSEQVYWS
LPDMMQDPVT FRSTRQISNF PVVGAELLPV HSKSFYNDQA
VYSQLIRQFT SLTHVFNRFP ENQILARPPA PTITTVSENV
PALTDHGTLP LRNSIGGVQR VTITDARRRT CPYVYKALGI
VSPRVLSSRT F
hAd7 (Q9JFT6); SEQ ID NO: 5:
MRRRAVLGGA MVYPEGPPPS YESVMQQQAA MIQPPLEAPF
VPPRYLAPTE GRNSIRYSEL SPLYDTTKLY LVDNKSADIA
SLNYQNDHSN FLTTVVQNND FTPTEASTQT INFDERSRWG
GQLKTIMHTN MPNVNEYMFS NKFKARVMVS RKAPEGVIVN
DTYDHKEDIL KYEWFEFTLP EGNFSATMTI DLMNNAIIDN
YLEIGRQNGV LESDIGVKFD TRNFRLGWDP ETKLIMPGVY
TYEAFHPDIV LLPGCGVDFT ESRLSNLLGI RKRHPFQEGF
KIMYEDLEGG NIPALLDVTA YEESKKDTTT ETTTLAVAEE
TSEDDNITRG DTYITEKQKR EAAAAEVKKE LKIQPLEKDS
KSRSYNVLED KINTAYRSWY LSYNYGNPEK GIRSWTLLTT
SDVTCGAEQV YWSLPDMMQD PVTFRSTRQV NNYPVVGAEL
MPVFSKSFYN EQAVYSQQLR QATSLTHVFN RFPENQILIR
PPAPTITTVS ENVPALTDHG TLPLRSSIRG VQRVTVTDAR
RRTCPYVYKA LGIVAPRVLS SRTF
hAd11 (D2DM93); SEQ ID NO: 6:
MRRVVLGGAV VYPEGPPPSY ESVMQQQATA VMQSPLEAPF
VPPRYLAPTE GRNSIRYSEL APQYDTTRLY LVDNKSADIA
SLNYQNDHSN FLTTVVQNND FTPTEASTQT INFDERSRWG
GQLKTIMHTN MPNVNEYMFS NNFKARVMVS RKPPEGAAVG
DTYDHKQDIL EYEWFEFTLP EGNFSVTMTI DLMNNAIIDN
YLKVGRQNGV LESDIGVKFD TRNFKLGWDP ETKLIMPGVY
TYEAFHPDIV LLPGCGVDFT ESRLSNLLGI RKKQPFQEGF
KILYEDLEGG NIPALLDVDA YENSKKEQKA KIEAAAEAKA
NIVASDSTRV ANAGEVRGDN FAPTPVPTAE SLLADVSGGT
DVKLTIQPVE KDSKNRSYNV LEDKINTAYR SWYLSYNYGD
PEKGVRSWTL LTTSDVTCGA EQVYWSLPDM MQDPVTFRST
RQVSNYPVVG AELMPVFSKS FYNEQAVYSQ QLRQSTSLTH
VFNRFPENQI LIRPPAPTIT TVSENVPALT DHGTLPLRSS
IRGVQRVTVT DARRRTCPYV YKALGIVAPR VLSSRTF
hAd12 (P36716); SEQ ID NO: 7:
MRRAVELQTV AFPETPPPSY ETVMAAAPPY VPPRYLGPTE
GRNSIRYSEL SPLYDTTRVY LVDNKSSDIA SLNYQNDHSN
FLTTVVQNND YSPIEAGTQT INFDERSRWG GDLKTILHTN
MPNVNDFMFT TKFKARVMVA RKTNNEGQTI LEYEWAEFVL
PEGNYSETMT IDLMNNAIIE HYLRVGRQHG VLESDIGVKF
DTRNFRLGWD PETQLVTPGV YTNEAFHPDI VLLPGCGVDF
TESRLSNILG IRKRQPFQEG FVIMYEHLEG GNIPALLDVK
KYENSLQDQN TVRGDNFIAL NKAARIEPVE TDPKGRSYNL
LPDKKNTKYR SWYLAYNYGD PEKGVRSWTL LTTPDVTGGS
EQVYWSLPDM MQDPVTFRSS RQVSNYPVVA AELLPVHAKS
FYNEQAVYSQ LIRQSTALTR VFNRFPENQI LVRPPAATIT
TVSENVPALT DHGTLPLRSS ISGVQRVTIT DARRRTCPYV
YKALGIVSPR VLSSRTF
hAd17 (F1DT65); SEQ ID NO: 8:
MRRAVVSSSP PPSYESVMAQ ATLEVPFVPP RYMAPTEGRN
SIRYSELAPL YDTTRVYLVD NKSADIASLN YQNDHSNFLT
TVVQNNDFTP AEASTQTINF DERSRWGGDL KTILHTNMPN
VNEYMFTSKF KARVMVARKH PQGVEATDLS KDILEYEWFE
FTLPEGNFSE TMTIDLMNNA ILENYLQVGR QNGVLESDIG
VKFDSRNFKL GWDPVTKLVM PGVYTYEAFH PDVVLLPGCG
VDFTESRLSN LLGIRKKQPF QEGFRIMYED LEGGNIPALL
DVPKYLESKK KLEEALENAA KANGPARGDS SVSREVEKAA
EKELVIEPIK QDDSKRSYNL IEGTMDTLYR SWYLSYTYGD
PEKGVQSWTL LTTPDVTCGA EQVYWSLPDL MQDPVTFRST
QQVSNYPVVG AELMPFRAKS FYNDLAVYSQ LIRSYTSLTH
VFNRFPDNQI LCRPPAPTIT TVSENVPALT DHGTLPLRSS
IRGVQRVTVT DARRRTCPYV YKALGIVAPR VLSSRTF
hAd25 (M0QUK0); SEQ ID NO: 9:
MRRAVVSSSP PPSYESVMAQ ATLEVPFVPP RYMAPTEGRN
SIRYSELAPQ YDTTRVYLVD NKSADIASLN YQNDHSNFLT
TVVQNNDFTP AEASTQTINF DERSRWGGDL KTILHTNMPN
VNEYMFTSKF KARVMVARKH PENVDKTDLS QDKLEYEWFE
FTLPEGNFSE TMTIDLMNNA ILENYLQVGR QNGVLESDIG
VKFDSRNFKL GWDPVTKLVM PGVYTYEAFH PDVVLLPGCG
VDFTESRLSN LLGIRKKQPF QEGFRIMYED LEGGNIPALL
DTKKYLDSKK ELEDAAKEAA KQQGDGAVTR GDTHLTVAQE
KAAEKELVIV PIEKDESNRS YNLIKDTHDT MYRSWYLSYT
YGDPEKGVQS WTLLTTPDVT CGAEQVYWSL PDLMQDPVTF
RSTQQVSNYP VVGAELMPFR AKSFYNDLAV YSQLIRSYTS
LTHVFNRFPD NQILCRPPAP TITTVSENVP ALTDHGTLPL
RSSIRGVQRV TVTDARRRTC PYVYKALGIV APRVLSSRTF
hAd35 (Q7T941); SEQ ID NO: 10:
MRRVVLGGAV VYPEGPPPSY ESVMQQQQAT AVMQSPLEAP
FVPPRYLAPT EGRNSIRYSE LAPQYDTTRL YLVDNKSADI
ASLNYQNDHS NFLTTVVQNN DFTPTEASTQ TINFDERSRW
GGQLKTIMHT NMPNVNEYMF SNKFKARVMV SRKPPDGAAV
DTYDHKQDI LEYEWFEFTL PEGNFSVTMT IDLMNNAIID
NYLKVGRQNG VLESDIGVKF DTRNFKLGWD PETKLIMPGV
YTYEAFHPDI VLLPGCGVDF TESRLSNLLG IRKKQPFQEG
FKILYEDLEG GNIPALLDVD AYENSKKEQK AKIEAATAAA
EAKANIVASD STRVANAGEV RGDNFAPTPV PTAESLLADV
SEGTDVKLTI QPVEKDSKNR SYNVLEDKIN TAYRSWYLSY
NYGDPEKGVR SWTLLTTSDV TCGAEQVYWS LPDMMKDPVT
FRSTRQVSNY PVVGAELMPV FSKSFYNEQA VYSQQLRQST
SLTHVFNRFP ENQILIRPPA PTITTVSENV PALTDHGTLP
LRSSIRGVQR VTVTDARRRT CPYVYKALGI VAPRVLSSRT F
hAd37 (Q912J1); SEQ ID NO: 11
MRRAVVSSSP PPSYESVMAQ ATLEVPFVPP RYMAPTEGRN
SIRYSELAPL YDTTRVYLVD NKSADIASLN YQNDHSNFLT
TVVQNNDFTP AEASTQTINF DERSRWGGDL KTILHTNMPN
VNEYMFTSKF KARVMVARKK AEGADANDRS KDILEYQWFE
FTLPEGNFSE TMTIDLMNNA ILENYLQVGR QNGVLESDIG
VKFDSRNFKL GWDPVTKLVM PGVYTYEAFH PDVVLLPGCG
VDFTESRLSN LLGIRKKQPF QEGFRIMYED LVGGNIPALL
NVKEYLKDKE EAGKADANTI KAQNDAVPRG DNYASAAEAK
AAGKEIELKA ILKDDSDRSY NVIEGTTDTL YRSWYLSYTY
GDPEKGVQSW TLLTTPDVTC GAEQVYWSLP DLMQDPVTFR
STQQVSNYPV VGAELMPFRA KSFYNDLAVY SQLIRSYTSL
THVFNRFPDN QILCRPPAPT ITTVSENVPA LTDHGTLPLR
SSIRGVQRVT VTDARRRTCP YVYKALGIVA PRVLSSRTF
hAd41 (F8WQN4); SEQ ID NO: 12:
MRRAVGVPPV MAYAEGPPPS YESVMGSADS PATLEALYVP
PRYLGPTEGR NSIRYSELAP LYDTTRVYLV DNKSADIASL
NYQNDHSNFQ TTVVQNNDFT PAEAGTQTIN FDERSRWGAD
LKTILRTNMP NINEFMSTNK FKARLMVEKK NKETGLPRYE
WFEFTLPEGN YSETMTIDLM NNAIVDNYLE VGRQNGVLES
DIGVKFDTRN FRLGWDPVTK LVMPGVYTNE AFHPDIVLLP
GCGVDFTQSR LSNLLGIRKR LPFQEGFQIM YEDLEGGNIP
ALLDVTKYEA SIQKAKEEGK EIGDDTFATR PQDLVIEPVA
KDSKNRSYNL LPNDQNNTAY RSWFLAYNYG DPNKGVQSWT
LLTTADVTCG SQQVYWSLPD MMQDPVTFRP STQVSNYPVV
GVELLPVHAK SFYNEQAVYS QLIRQSTALT HVFNRFPENQ
ILVRPPAPTI TTVSENVPAL TDHGTLPLRS SISGVQRVTI
TDARRRTCPY VHKALGIVAP KVLSSRTF
Gorilla Adenovirus Penton Base gorAd (E5L3Q9);
SEQ ID NO: 13:
MMRRAVLGGA VVYPEGPPPS YESVMQQQAA AVMQPSLEAP
FVPPRYLAPT EGRNSIRYSE LAPQYDTTRL YLVDNKSADI
ASLNYQNDHS NFLTTVVQNN DFTPTEASTQ TINFDERSRW
GGQLKTIMHT NMPNVNEYMF SNKFKARVMV SREASKIDSE
KNDRSKDTLK YEWFEFTLPE GNFSATMTID LMNNAIIDNY
LAVGRQNGVL QSDIGVKFDT RNFRLGWDPV TKLVMPGVYT
YEAFHPDIVL LPDCGVDFTE SRLSNLLGIR KRHPFQEGFK
IMYEDLEGGN IPALLDVAEY EKSKKEIASS TTTTAVTTVA
RNVADTSVEA VAVAVVDTIK AENDSAVRGD NFQSKNDMKA
SEEVTVVPVS PPTVTETETK EPTIKPLEKD TKDRSYNVIS
GTNDTAYRSW YLAYNYGDPE KGVRSWTLLT TSDVTCGAEQ
VYWSLPDMMQ DPVTFRSTRQ VSNYPVVGAE LMPVFSKSFY
NEQAVYSQQL RQTTSLTHIF DRFPENQILI RPPAPTITTV
SENVPALTDH GTLPLRSSIR GVQRVTVTDA RRRTCPYVYK
ALGIVAPRVL SSRTF
Cimpanzee Adenovirus Penton Base chimpAd
(G9G849); SEQ ID NO: 14:
MMRRAYPEGP PPSYESVMQQ AMAAAAAMQP PLEAPYVPPR
YLAPTEGRNS IRYSELAPLY DTTRLYLVDN KSADIASLNY
QNDHSNFLTT VVQNNDFTPT EASTQTINFD ERSRWGGQLK
TIMHTNMPNV NEFMYSNKFK ARVMVSRKTP NGVTVTDGSQ
DILEYEWVEF ELPEGNFSVT MTIDLMNNAI IDNYLAVGRQ
NGVLESDIGV KFDTRNFRLG WDPVTELVMP GVYTNEAFHP
DIVLLPGCGV DFTESRLSNL LGIRKRQPFQ EGFQIMYEDL
EGGNIPALLD VDAYEKSKEE SAAAATAAVA TASTEVRGDN
FASPAAVAAA EAAETESKIV IQPVEKDSKD RSYNVLPDKI
NTAYRSWYLA YNYGDPEKGV RSWTLLTTSD VTCGVEQVYW
SLPDMMQDPV TFRSTRQVSN YPVVGAELLP VYSKSFFNEQ
AVYSQQLRAF TSLTHVFNRF PENQILVRPP APTITTVSEN
VPALTDHGTL PLRSSIRGVQ RVTVTDARRR TCPYVYKALG
IVAPRVLSSR TF
Simian Adenovirus Serotype 18 Penton Base,
sAd18 (H8PFZ9); SEQ ID NO: 15:
MRRAVGVPPV MAYAEGPPPS YETVMGAADS PATLEALYVP
PRYLGPTEGR NSIRYSELAP LYDTTRVYLV DNKSADIASL
NYQNDHSNFL TTVVQNNDFT PVEAGTQTIN FDERSRWGGD
LKTILRTNMP NINEFMSTNK FRARLMVEKV NKETNAPRYE
WFEFTLPEGN YSETMTIDLM NNAIVDNYLE VGRQNGVLES
DIGVKFDTRN FRLGWDPVTK LVMPGVYTNE AFHPDIVLLP
GCGVDFTQSR LSNLLGIRKR MPFQAGFQIM YEDLEGGNIP
ALLDVAKYEA SIQKAREQGQ EIRGDNFTVI PRDVEIVPVE
KDSKDRSYNL LPGDQTNTAY RSWFLAYNYG DPEKGVRSWT
LLTTTDVTCG SQQVYWSLPD MMQDPVTFRP SSQVSNYPVV
GVELLPVHAK SFYNEQAVYS QLIRQSTALT HVFNRFPENQ
ILVRPPAPTI TTVSENVPAL TDHGTLPLRS SISGVQRVTI
TDARRRTCPY VHKALGIVAP KVLSSRTF
sAd20 (F6KSU4); SEQ ID NO: 16:
MRRAVAIPSA AVALGPPPSY ESVMASANLQ APLENPYVPP
RYLEPTGGRN SIRYSELTPL YDTTRLYLVD NKSADIATLN
YQNDHSNFLT SVVQNSDYTP AEASTQTINL DDRSRWGGDL
KTILHTNMPN VNEFMFTNSF RAKLMVAHET NKDPVYKWVE
LTLPEGNFSE TMTIDLMNNA IVDHYLAVGR QNGVKESEIG
VKFDTRNFRL GWDPQTELVM PGVYTNEAFH PDVVLLPGCG
VDFTYSRLSN LLGIRKRMPF QEGFQIMYED LVGGNIPALL
DVPAYEASIT TVAAKEVRGD NFEAAAAAAA TGAQPQAAPV
VRPVTQDSKG RSYNIITGTN NTAYRSWYLA YNYGDPEKGV
RSWTLLTTPD VTCGSEQVYW SMPDMYVDPV TFRSSQQVSS
YPVVGAELLP IHSKSFYNEQ AVYSQLIRQQ TALTHVFNRF
PENQILVRPP APTITTVSEN VPALTDHGTL PLQNSIRGVQ
RVTITDARRR TCPYVYKALG IVAPRVLSSR TF
sAd49 (F2WTK5); SEQ ID NO: 17:
MRRAVPAAAI PATVAYADPP PSYESVMAGV PATLEAPYVP
PRYLGPTEGR NSIRYSELAP LYDTTRVYLV DNKSADIASL
NYQNDHSNFL TTVVQNNDFT PVEAGTQTIN FDERSRWGGQ
LKTILHTNMP NVNEFMFTNS FRAKVMVSRK QNEEGQTELE
YEWVEFVLPE GNYSETMTLD LMNNAIVDHY LLVGRQNGVL
ESDIGVKFDT RNFRLGWDPV TKLVMPGVYT NEAFHPDVVL
LPGCGVDFTQ SRLSNLLGIR KRQPFQEGFR IMYEDLEGGN
IPALLNVKAY EDSIAAAMRK HNLPLRGDVF AVQPQEIVIQ
PVEKDGKERS YNLLPDDKNN TAYRSWYLAY NYGDPLKGVR
SWTLLTTPDV TCGSEQVYWS LPDLMQDPVT FRPSSQVSNY
PVVGAELLPL QAKSFYNEQA VYSQLIRQST ALTHVFNRFP
ENQILVRPPA ATITTVSENV PALTDHGTLP LRSSISGVQR
VTITDARRRT CPYVYKALGI VAPRVLSSRT F
Rhesus Adenovirus Serotype 51 Penton Base,
rhAd51 (A0A0A1EWW1); SEQ ID NO: 18:
MRRAVRVTPA AYEGPPPSYE SVMGSANVPA TLEAPYVPPR
YLGPTEGRNS IRYSELAPLY DTTKVYLVDN KSADIASLNY
QNDHSNFLTT VVQNNDFTPT EAGTQTINFD ERSRWGGQLK
TILHTNMPNI NEFMSTNKFR AKLMVEKSNA ETRQPRYEWF
EFTIPEGNYS ETMTIDLMNN AIVDNYLQVG RQNGVLESDI
GVKFDTRNFR LGWDPVTKLV MPGVYTNEAF HPDIVLLPGC
GVDFTQSRLS NLLGIRKRRP FQEGFQIMYE DLEGGNIPAL
LDVSKYEASI QRAKAEGREI RGDTFAVAPQ DLEIVPLTKD
SKDRSYNIIN NTTDTLYRSW FLAYNYGDPE KGVRSWTILT
TTDVTCGSQQ VYWSLPDMMQ DPVTFRPSTQ VSNFPVVGTE
LLPVHAKSFY NEQAVYSQLI RQSTALTHVF NRFPENQILV
RPPAPTITTV SENVPALTDH GTLPLRSSIS GVQRVTITDA
RRRTCPYVYK ALGVVAPKVL SSRTF
rhAd52 (A0A0A1EWX7); SEQ ID NO: 19:
MRRAVRVTPA AYEGPPPSYE SVMGSANVPA TLEAPYVPPR
YLGPTEGRNS IRYSELAPLY DTTKVYLVDN KSADIASLNY
QNDHSNFLTT VVQNNDFTPT EAGTQTINFD ERSRWGGQLK
TILHTNMPNI NEFMSTNKFR ARLMVKKVEN QPPEYEWFEF
TIPEGNYSET MTIDLMNNAI VDNYLQVGRQ NGVLESDIGV
KFDTRNFRLG WDPVTKLVMP GVYTNEAFHP DIVLLPGCGV
DFTQSRLSNL LGIRKRRPFQ EGFQIMYEDL EGGNIPALLD
VTKYEQSVQR AKAEGREIRG DTFAVSPQDL VIEPLEHDSK
NRSYNLLPNK TDTAYRSWFL AYNYGDPEKG VRSWTILITT
DVTCGSQQVY WSLPDMMQDP VTFRPSTQVS NFPVVGTELL
PVHAKSFYNE QAVYSQLIRQ STALTHVFNR FPENQILVRP
PAPTITTVSE NVPALTDHGT LPLRSSISGV QRVTITDARR
RTCPYVYKAL GVVAPKVLSS RTF
rhAd53 (A0A0A1EWZ7); SEQ ID NO: NO 20:
MRRAVRVTPA VYAEGPPPSY ESVMGSANVP ATLEAPYVPP
RYLGPTEGRN SIRYSELAPL YDTTKVYLVD NKSADIASLN
YQNDHSNFLT TVVQNNDFTP TEAGTQTINF DERSRWGGQL
KTILHTNMPN INEFMSTNKF RARLMVEKTS GQPPKYEWFE
FTIPEGNYSE TMTIDLMNNA IVDNYLQVGR QNGVLESDIG
VKFDTRNFRL GWDPVTKLVM PGVYTNEAFH PDIVLLPGCG
VDFTQSRLSN LLGIRKRRPF QEGFQIMYED LEGGNIPGLL
DVPAYEQSLQ QAQEEGRVTR GDTFATAPNE VVIKPLLKDS
KDRSYNIITD TTDTLYRSWF LAYNYGDPEN GVRSWTILTT
TDVTCGSQQV YWSLPDMMQD PVTFRPSTQV SNFPVVGTEL
LPVHAKSFYN EQAVYSQLIR QSTALTHVFN RFPENQILVR
PPAPTITTVS ENVPALTDHG TLPLRSSISG VQRVTITDAR
RRTCPYVYKA LGVVAPKVLS SRTF

The polypeptide of the present invention is not confined to those known specific sequences for amino acid stretches A, B, and C forming the multimerization jellyroll fold domain of the above-referenced adenovirus sub- and serotypes, respectively. Amino acid segments A, B, and C can also have similar amino acid sequences to the sequences of known adenovirus penton base protomers as long as the sequences of A, B, and C are such that the resulting polypeptide adopts the jellyroll fold and assembles into pentameric complexes (also denoted “penton proteins”) twelve of which in turn self-assemble to form a dodecameric supercomplex (the VLP of the invention) under appropriate conditions as further outlined below. Typically, such similar sequences of segments A, B and C share an amino acid sequence identity of at least 85%, more preferred at least 90%, even more preferred 95%, particularly preferred at least 98%, most preferred at least 99%, with the respective amino acid sequence of a known adenovirus penton base, preferably those of SEQ ID NOs: 1 to 20, more preferably amino acid stretches A, B and C as provided in below Tables 1 to 3.

As used herein, amino acid sequences are stated from N to C terminal using the single letter code of IUPAC, if not otherwise specifically indicated.

According to a preferred embodiment of the invention, amino acid stretch A has the following consensus sequence (SEQ ID NO: 21):

(U)₁₋₄₇PTX₁GRNSIRYSX₂X₃x₄PX₅X₆DTTX₇X₈YLVDNKSADIASL
NYQNDHSNFX₅TTVX₉QNNDX₁₀X₁₁PX₁₂EAX₁₃TQTINX₁₄DX₁₅RSR
WGX₁₆X₁₇LKTIX₁₈X₁₉TZ₁Z₂Z₃Z₄Z₅Z₆Z₇Z₈Z₉Z₁₀Z₁₁Z₁₂Z₁₃
Z₁₄Z₁₅

wherein: amino acid stretch A ends on the C-terminal side before Z₁ at residue T or at an amino acid from Z₁ to Z₁₅
U is any or no amino acid
X₁ is E or G
X₂ is E or S
X₃ is L or V
X₄ is A or S
X₅ is L or Q
X₆ is Y or E
X₇ is R or K
X₈ is V or L
X₉ is V or I
X₁₀ is F or Y
X₁₁ is T or S
X₁₂ is A or T or I or G
X₁₃ is S or G
X₁₄ is F or L
X₁₅ is E or D
X₁₆ is A or G
X₁₇ is D or Q
X₁₈ is L or M
X₁₉ is H or R
Z₁, if present, is N
Z₂, if present, is M
Z₃, if present, is P
Z₄, if present, is N
Z₅, if present, is V or I
Z₆, if present, is N
Z₇, if present, is E or D
Z₈, if present, is Y or F
Z₉, if present, is M
Z₁₀, if present, is F or S or Y
Z₁₁, if present, is T or S
Z₁₂, if present, is S or N
Z₁₃, if present, is K
Z₁₄, if present, is F
Z₁₆, if present, is K

More preferred amino acid sequences of segment A of the polypeptide according to the invention are outlined in the following Table 1:

TABLE 1
Preferred sequences for segment A of general formula (I)

| Sequence | Sequence | | N-terminal | C-terminal |
|---|---|---|---|---|
| based on | according | SEQ | amino acid | amino acid |
| penton base | to UniProt | ID | selected from | selected from |
| protomer of | Acc. No. | NO: | positions | positions |
| hAd3 | Q2Y0H9 | 1 | 1 to 48 | 129 to 144 |
| hAd2 | P03276 | 2 | 1 to 48 | 129 to 144 |
| hAd4 | Q2KSF3 | 3 | 1 to 44 | 125 to 140 |
| hAd5 | P12538 | 4 | 1 to 48 | 129 to 144 |
| hAd7 | Q9JFT6 | 5 | 1 to 48 | 129 to 144 |
| hAd11 | D2DM93 | 6 | 1 to 48 | 129 to 144 |
| hAd12 | P36716 | 7 | 1 to 38 | 119 to 134 |
| hAd17 | F1DT65 | 8 | 1 to 35 | 116 to131 |
| hAd25 | M0QUK0 | 9 | 1 to 43 | 124 to 139 |
| hAd35 | Q7T941 | 10 | 1 to 49 | 130 to 145 |
| hAd37 | Q912J1 | 11 | 1 to 35 | 116 to 131 |
| hAd41 | F8WQN4 | 12 | 1 to 46 | 127 to 142 |
| gorAd | E5L3Q9 | 13 | 1 to 49 | 130 to 145 |
| ChimpAd | G9G849 | 14 | 1 to 44 | 125 to 140 |
| sAd18 | H8PFZ9 | 15 | 1 to 46 | 127 to 142 |
| sAd20 | F6KSU4 | 16 | 1 to 45 | 126 to 141 |
| sAd49 | F2WTK5 | 17 | 1 to 48 | 127 to 142 |
| rhAd51 | A0A0A1EWW1 | 18 | 1 to 43 | 124 to 139 |
| rhAd52 | A0A0A1EWX7 | 19 | 1to 43 | 124 to 139 |
| rhAd53 | A0A0A1EWZ7 | 20 | 1 to 44 | 125 to 140 |

According to a further preferred embodiment of the invention, amino acid stretch B of above general formula (I) has the following sequence (SEQ ID NO: 22):

Z₁₇Z₁₈Z₁₉Z₂₀Z₂₁Z₂₂Z₂₃ZZ₂₄ZZ₂₅Z₂₆Z₂₇QVYWSLPDX₂₀MX₂₁
DPVTFRSTX₂₂QX₂₃X₂₄NX₂₅PVVGX₂₆ELZ₂₈Z₂₉Z₃₀

wherein: amino acid stretch B begins on the N-terminal side at an amino acid from Z₁₇ to Z₂₇ or at amino acid Q after Z₂₇;
amino acid stretch B ends on the C-terminal side before Z₂₈ at amino acid L or at an amino acid from Z₂₈ to Z₃₀;
Z₁₇, if present, is L or S
Z₁₈, if present, is T or P or C
Z₁₉, if present, is T or P
Z₂₀, if present, is P or S or A or R
Z₂₁, if present, is N or D
Z₂₂, if present, is G or V
Z₂₃, if present, is H or T
Z₂₄, if present, is C
Z₂₅, if present, is G
Z₂₈, if present, is A or V or S
Z₂₇, if present, is E or Q
X₂₀ is L or M
X₂₁ is Q or K
X₂₂ is Q or R or S
X₂₃ is V or I
X₂₄ is S or N
X₂₅ is Y or F
X₂₈ is A or V
Z₂₈, if present, is M or L
Z₂₉, if present, is P
Z₃₀, if present, is V or F

More preferred amino acid sequences of segment B of the polypeptide according to the invention are outlined in the following Table 2:

TABLE 2
Preferred sequences for segment B of general formula (I)

| Sequence | Sequence | | N-terminal | C-terminal |
|---|---|---|---|---|
| based on | according | SEQ | amino acid | amino acid |
| penton base | to UniProt | ID | selected from | selected from |
| protomer of | Acc. No. | NO: | positions | positions |
| hAd3 | Q2Y0H9 | 1 | 398 to 409 | 440 to 443 |
| hAd2 | P03276 | 2 | 425 to 436 | 467 to 470 |
| hAd4 | Q2KSF3 | 3 | 379 to 390 | 421 to 444 |
| hAd5 | P12538 | 4 | 425 to 436 | 467 to 470 |
| hAd7 | Q9JFT6 | 5 | 398 to 409 | 440 to 443 |
| hAd11 | D2DM93 | 6 | 415 to 426 | 457 to 460 |
| hAd12 | P36716 | 7 | 351 to 362 | 393 to 397 |
| hAd17 | F1DT65 | 8 | 370 to 381 | 413 to 416 |
| hAd25 | M0QUK0 | 9 | 388 to 399 | 440 to 443 |
| hAd35 | Q7T941 | 10 | 445 to 456 | 497 to 500 |
| hAd37 | Q912J1 | 11 | 372 to 383 | 414 to 417 |
| hAd41 | F8WQN4 | 12 | 362 to 373 | 404 to 407 |
| gorAd | E5L3Q9 | 13 | 416 to 427 | 458 to 461 |
| ChimpAd | G9G849 | 14 | 372 to 383 | 420 to 423 |
| sAd18 | H8PFZ9 | 15 | 353 to 364 | 395 to 398 |
| sAd20 | F6KSU4 | 16 | 358 to 369 | 400 to 403 |
| sAd49 | F2WTK5 | 17 | 356 to 367 | 398 to 401 |
| rhAd51 | A0A0A1EWW1 | 18 | 352 to 363 | 394 to 397 |
| rhAd52 | A0A0A1EWX7 | 19 | 350 to 361 | 392 to 395 |
| rhAd53 | A0A0A1EWZ7 | 20 | 351 to 362 | 393 to 396 |

According to a further preferred embodiment of the invention, segment C of above general formula (I) has the following sequence (SEQ ID NO: 23):

Z₃₁Z₃₂Z₃₃ALTDHGTLPLRSSIX₂₇GVQRVTX₂₈TDARRRTCPYVYKA
LGIVX₃₀PX₃₁VLSSRTF

wherein: amino acid stretch C begins on the N-terminal side at an amino acid from Z₃₁ to Z₃₃ or at amino acid A after Z₃₃;
Z₃₁, if present, is N
Z₃₂, if present, is V
Z₃₃, if present, is P
X₂₇ is R or S or G
X₂₈ is V or I
X₂₉ is Y or H
X₃₀ is A or S
X₃₁ is R or K

More preferred amino acid sequences of segment C of the polypeptide according to the invention are outlined in the following Table 3:

TABLE 3
Preferred sequences for segment C of general formula (I)

| Sequence | Sequence | | N-terminal | |
|---|---|---|---|---|
| based on | according | SEQ | amino acid | C-terminal |
| penton base | to UniProt | ID | selected from | amino acid |
| protomer of | Acc. No. | NO: | positions | position |
| hAd3 | Q2Y0H9 | 1 | 492 to 495 | 544 |
| hAd2 | P03276 | 2 | 519 to 522 | 571 |
| hAd4 | Q2KSF3 | 3 | 466 to 469 | 535 |
| hAd5 | P12538 | 4 | 492 to 495 | 571 |
| hAd7 | Q9JFT6 | 5 | 465 to 468 | 544 |
| hAd11 | D2DM93 | 6 | 482 to 485 | 561 |
| hAd12 | P36716 | 7 | 419 to 422 | 497 |
| hAd17 | F1DT65 | 8 | 438 to 441 | 517 |
| hAd25 | M0QUK0 | 9 | 455 to 458 | 534 |
| hAd35 | Q7T941 | 10 | 522 to 525 | 561 |
| hAd37 | Q912J1 | 11 | 439 to 442 | 519 |
| hAd41 | F8WQN4 | 12 | 439 to 432 | 508 |
| gorAd | E5L3Q9 | 13 | 483 to 486 | 875 |
| ChimpAd | G9G849 | 14 | 445 to 458 | 532 |
| sAd18 | H8PFZ9 | 15 | 420 to 423 | 508 |
| sAd20 | F6KSU4 | 16 | 425 to 428 | 512 |
| sAd49 | F2WTK5 | 17 | 423 to 426 | 511 |
| rhAd51 | A0A0A1EWW1 | 18 | 419 to 422 | 505 |
| rhAd52 | A0A0A1EWX7 | 19 | 417 to 420 | 503 |
| rhAd53 | A0A0A1EWZ7 | 20 | 418 to 421 | 504 |

Particularly preferred polypeptides of the invention are based on the jellyroll fold domain of the penton base protomer of hAd3. In particular, polypeptides are preferred wherein amino acid stretch A has an amino acid sequence starting at a position selected from amino acids 1 to 48, most preferred amino acid position 1, until an amino acid position selected from positions 129 to 144, most preferred amino acid position 132, amino acid stretch B has an amino acid sequence starting at a position selected from position 398 to 409, most preferred amino acid position 407, until a position selected from positions 440 to 443, most preferred amino acid position 442, and amino acid stretch C has an amino acid sequence starting at a position selected from position 492 to 495, most preferred amino acid position 493, until amino acid position 544, wherein amino acid positions refer to the sequence laid down in UniProt Acc. No. QY0H9 (SEQ ID NO: 1).

The linking segments L1 and L2 of the polypeptide according to the invention may be selected from oligopeptide linkers such as oligopeptides having 4 to 10 amino acids, preferably having amino acids G and S. A preferred example is GGGS (SEQ ID 24). Another example is a linker composed of G and S and having multiple GGS repeats such as 2, 3, 4, 5 or more GGS repeats. A particularly preferred linker of this type id GGSGGS (SEQ ID NO: 25).

In other preferred embodiments, L₁ is a polypeptide sequence comprising an RGD loop of an adenovirus penton base having a different serotype compared to the serotype of the adenovirus(es) from which said amino acid stretches A. B and C are derived and/or L₂ is a polypeptide sequence comprising a variable loop of an adenovirus penton base having a different serotype compared to the serotype of the adenovirus from which said amino acid stretches A, B and C are derived.

In further embodiments of the invention L₁ is an RDG loop and L₂ is a, preferably non-adenoviral oligopeptide of preferably 4 to 20 amino acids, more preferably 4 to 10 amino acids, particularly preferred an oligopeptide linker composed of G and S as defined above. In similar embodiments, L₂ is or comprises a variable loop, and L₁ is an oligopeptide linker as defined before. It is also envisaged according to the invention that L₂ is or comprises an RGD loop and L₁ is an oligopeptide linker, and it is also contemplated that L₁ is a variable loop and L₂ is an oligopeptide linker.

In other preferred embodiments, as mentioned before, the L₁ and L₂, respectively sequences may be selected from crown domain sequences of penton base proteins from an adenovirus other than the adenovirus from which the multimerization domain is derived. Generally, the combination of the crown-multimerization domain chimera is not restricted. Preferred chimeras are selected from combinations of the crown and multimerization domains as outlined above. The crown domains, optionally, and preferably, including non-adenoviral sequences inserted in an RGD loop and/or a variable loop of the respective crown domain, are more preferably as disclosed in WO 2017/167988 A1.

It is thereby understood that crown domains of adenovirus penton bases are typically made up of two amino acid stretches: the so-called big fragment and small fragment. The big fragment of the crown domain is located more N-terminally in the amino acid sequence of the respective adenovirus penton base protein whereas the small fragment of the crown domain is located more C-terminally. According to the invention it is preferred when the big fragment (containing the RGD loop as mentioned above) corresponds to L₁ of general formula (I), and it is further preferred that the small fragment (containing the variable loop) corresponds to L₂ of general formula (I). According to certain embodiments of the invention, the big and small fragment stem from the same adenovirus penton base. According to other embodiments of the invention, the big fragment and the small fragment stem from different adenovirus penton bases, or that only one of the big and small fragments stem from an adenovirus penton base protein different from the adenovirus from which the multimerization domain, i.e. amino stretches A, B and C) is derived from.

Preferred crown domains for use in the chimeric constructs of the invention include the crown domains selected from the group consisting of from the group consisting of penton bases of human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53).

Preferred amino acid sequences of the above-indicated adenovirus penton bases used for the crown domain are laid down in generally accessible databases such as UniProt and UniProtE, and especially preferred sequences referred to herein for the above-mentioned adenovirus subtypes are laid down in UniProtAcc. No. Q2Y0H9 (human adenovirus serotype 3; SEQ ID NO: 1), UniProt Acc. No. P03276 (human adenovirus serotype 2; SEQ ID NO: 2), UniProt Acc. No. Q2KSF3 (human adenovirus serotype 4; SEQ ID NO: 3), UniProt Acc. No. P12538 (human adenovirus serotype 5; SEQ ID NO: 4), UniProt Acc. No. Q9JFT6 (human adenovirus serotype 7; SEQ ID NO: 5), UniProt Acc. No. D2DM93 (human adenovirus serotype 11; SEQ ID NO: 6), UniProtAcc. No. P36716 (human adenovirus serotype 12; SEQ ID NO: 7), UniProt Acc. No. F1DT65 (human adenovirus serotype 17; SEQ ID NO: 8), UniProt Acc. No. M0QUK0 (human adenovirus serotype 25; SEQ ID NO: 9), UniProt Acc. No. Q7T941 (human adenovirus serotype 35; SEQ ID NO: 10), UniProtAcc. No. Q912J1 (human adenovirus serotype 37; SEQ ID NO: 11), UniProt Acc. No. F8WQN4 (human adenovirus serotype 41; SEQ ID NO: 12), UniProt Acc. No. E5L3Q9 (gorilla adenovirus; SEQ ID NO: 13), UniProt Acc. No. G9G849 (chimpanzee adenovirus; SEQ ID NO: 14), UniProt Acc. No. H8PFZ9 (simian adenovirus serotype 18; SEQ ID NO: 15), UniProt Acc. No. F6KSU4 (simian adenovirus serotype 20; SEQ ID NO: 16), UniProt Acc. No. F2WTK5 (simian adenovirus serotype 49; SEQ ID NO: 17), UniProt Acc. No. A0A0A1EWW1 (rhesus adenovirus serotype 51; SEQ ID NO: 18), UniProt Acc. No. A0A0A1EWX7 (rhesus adenovirus serotype 52; SEQ ID NO: 19), and UniProt Acc. No. A0A0A1EWZ7 (rhesus adenovirus serotype 53; SEQ ID NO: 20).

Most preferred sequences of big fragments of crown domains for use in the chimeric constructs of the invention are outlined in the following Table 4:

TABLE 4
Preferred sequences for big fragment of crown
domains for use in chimeric constructs

| Sequence | Sequence | | N-terminal | C-terminal |
|---|---|---|---|---|
| based on | according | SEQ | amino acid | amino acid |
| penton base | to UniProt | ID | selected from | selected from |
| protomer of | Acc. No. | NO: | positions | positions |
| hAd3 | Q2Y0H9 | 1 | 130 to 145 | 397 to 408 |
| hAd2 | P03276 | 2 | 130 to 145 | 424 to 435 |
| hAd4 | Q2KSF3 | 3 | 126 to 141 | 378 to 389 |
| hAd5 | P12538 | 4 | 130 to 145 | 424 to 435 |
| hAd7 | Q9JFT6 | 5 | 130 to 145 | 397 to 408 |
| hAd11 | D2DM93 | 6 | 130 to 145 | 414 to 425 |
| hAd12 | P36716 | 7 | 120 to 135 | 350 to 361 |
| hAd17 | F1DT65 | 8 | 117 to 132 | 369 to 380 |
| hAd25 | M0QUK0 | 9 | 125 to 140 | 387 to 398 |
| hAd35 | Q7T941 | 10 | 131 to 146 | 444 to 455 |
| hAd37 | Q912J1 | 11 | 117 to 132 | 371 to 382 |
| hAd41 | F8WQN4 | 12 | 128 to 143 | 361 to 373 |
| gorAd | E5L3Q9 | 13 | 131 to 146 | 415 to 427 |
| ChimpAd | G9G849 | 14 | 126 to 141 | 371 to 382 |
| sAd18 | H8PFZ9 | 15 | 128 to 143 | 352 to 363 |
| sAd20 | F6KSU4 | 16 | 127 to 142 | 357 to 368 |
| sAd49 | F2WTK5 | 17 | 128 to 143 | 355 to 366 |
| rhAd51 | A0A0A1EWW1 | 18 | 125 to 140 | 351 to 362 |
| rhAd52 | A0A0A1EWX7 | 19 | 125 to 140 | 349 to 360 |
| rhAd53 | A0A0A1EWZ7 | 20 | 126 to 141 | 350 to 361 |

Most preferred sequences of big fragments of crown domains for use in the chimeric constructs of the invention are outlined in the following Table 5:

TABLE 5
Preferred sequences for big fragment of crown
domains for use in chimeric constructs

| Sequence | Sequence | | N-terminal | C-terminal |
|---|---|---|---|---|
| based on | according | SEQ | amino acid | amino acid |
| penton base | to UniProt | ID | selected from | selected from |
| protomer of | Acc. No. | NO: | positions | positions |
| hAd3 | Q2Y0H9 | 1 | 441 to 444 | 491 to 494 |
| hAd2 | P03276 | 2 | 468 to 471 | 518 to 521 |
| hAd4 | Q2KSF3 | 3 | 422 to 445 | 465 to 468 |
| hAd5 | P12538 | 4 | 468 to 471 | 491 to 494 |
| hAd7 | Q9JFT6 | 5 | 441 to 444 | 464 to 467 |
| hAd11 | D2DM93 | 6 | 458 to 461 | 481 to 484 |
| hAd12 | P36716 | 7 | 394 to 398 | 418 to 421 |
| hAd17 | F1DT65 | 8 | 414 to 417 | 438 to 441 |
| hAd25 | M0QUK0 | 9 | 441 to 444 | 454 to 457 |
| hAd35 | Q7T941 | 10 | 498 to 501 | 521 to 524 |
| hAd37 | Q912J1 | 11 | 415 to 418 | 438 to 441 |
| hAd41 | F8WQN4 | 12 | 405 to 408 | 438 to 441 |
| gorAd | E5L3Q9 | 13 | 459 to 462 | 482 to 485 |
| ChimpAd | G9G849 | 14 | 421 to 424 | 444 to 457 |
| sAd18 | H8PFZ9 | 15 | 396 to 399 | 419 to 422 |
| sAd20 | F6KSU4 | 16 | 401 to 404 | 424 to 427 |
| sAd49 | F2WTK5 | 17 | 399 to 402 | 422 to 425 |
| rhAd51 | A0A0A1EWW1 | 18 | 395 to 398 | 418 to 421 |
| rhAd52 | A0A0A1EWX7 | 19 | 393 to 396 | 416 to 419 |
| rhAd53 | A0A0A1EWZ7 | 20 | 394 to 397 | 417 to 420 |

A preferred embodiment of the invention is a chimera in which a multimerization domain of human adenovirus serotype 2 (hAd2) is combined with a crown domain of an adenovirus penton base selected from human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 3 (hAd3) is combined with a crown domain of an adenovirus penton base selected from human adenovirus serotype 2 (hAd2), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 4 (hAd4) is combined with a crown domain of an adenovirus penton base selected from human adenovirus serotype 2 (hAd2), human adenovirus serotype 3 (hAd3), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 5 (hAd5) is combined with a crown domain of an adenovirus penton base selected from human adenovirus serotype 2 (hAd2), human adenovirus serotype 3 (hAd3), human adenovirus serotype 4 (hAd4), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 7 (hAd7) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 11 (hAd11) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 12 (hAd12) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 17 (hAd17) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 25 (hAd25) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 35 (hAd35) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 37 (hAd37) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of human adenovirus serotype 41 (hAd41) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of gorilla adenovirus (gorAd) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd41), human adenovirus serotype 41 (hAd41), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of chimpanzee adenovirus (ChimpAd) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd41), human adenovirus serotype 41 (hAd41), gorilla adenovirus (gorAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of simian adenovirus serotype 18 (sAd18) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd41), human adenovirus serotype 41 (hAd41), gorilla adenovirus (gorAd), chimpanzee adenovirus (ChimpAd), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of simian adenovirus serotype 20 (sAd20) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd41), human adenovirus serotype 41 (hAd41), gorilla adenovirus (gorAd), chimpanzee adenovirus (ChimpAd), simian adenovirus serotype 18 (sAd18), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of simian adenovirus serotype 49 (sAd49) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd41), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of rhesus adenovirus serotype 51 (rhAd51) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd41), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of rhesus adenovirus serotype 52 (rhAd52) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd41), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), and rhesus adenovirus serotype 53 (rhAd53). With respect to specific sequences for the multimerization and the crown domain selected for this combination it is referred to the specific examples according to Tables 1 to 5.

A preferred embodiment of the invention is a chimera in which a multimerization domain of rhesus adenovirus serotype 53 (rhAd53) is combined with a crown domain of an adenovirus penton base selected from human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd41), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), and rhesus adenovirus serotype 52 (rhAd52). With respect to specific sequences for the multimerization and the crown domain selected for this combination it is referred to the specific examples according to Tables 1 to 5.

A particularly preferred crown domain for providing chimeras of the invention is the crown domain of the penton base protein of human adenovirus serotype 3 (hAd3). For preferred sequences as regards the amino acid positions of SEQ ID NO: 1 it is referred to Table 4 (big fragment) and Table 5 (small fragment).

In even more preferred chimeras of the invention, the crown domain of the penton base protein of human adenovirus serotype 3 (hAd3) is combined with a multimerization domain of a penton base protein of an adenovirus selected from human adenovirus serotype 2 (hAd2), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

A particularly preferred crown domain for providing chimeras of the invention is the crown domain of the penton base of chimpanzee adenovirus (ChimpAd). For preferred sequences as regards the amino acid positions of SEQ ID NO: 14 it is referred to Table 4 (big fragment) and Table 5 (small fragment).

In even more preferred chimeras of the invention, the crown domain of the penton base protein of chimpanzee adenovirus (ChimpAd) is combined with a multimerization domain of a penton base protein of an adenovirus selected from human adenovirus serotype 3 (hAd3), human adenovirus serotype 2 (hAd2), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53). With respect to specific sequences for the multimerization and the crown domain selected for this combination it is referred to the specific examples according to Tables 1 to 5.

As already outlined above, it is one premier embodiment of the invention to include an antigen, more particularly an antigen of an infectious agent such as a virus, bacterium or other pathogen, or a tumour or cancer antigen, into one or both of L₁ and L₂. With respect to preferable sites of inclusion of antigens in RGD loops and/or variable loops of adenoviral crown domains, it is expressis verbis referred to WO 2017/167988 A1. As used herein, the term “antigen” refers a structure recognized by molecules of the immune response, e.g. antibodies, T cell receptors (TCRs) etc.

Antigens of infectious agents include, but are not limited to, e.g. viral infectious agents, such as HIV, hepatitis viruses such as hepatitis A virus, hepatitis B virus or hepatitis C virus, herpes virus, varicella zoster virus, rubella virus, yellow fever virus, dengue fever virus, flaviviruses (e.g. Zika virus), influenza viruses, Marburg disease virus, Ebola viruses and arboviruses such as Chikungunya virus. Antigens of bacterial infectious agents include, but are not limited to, antigens of e.g. Legionella, Helicobacter, Vibrio, infectious E. coli strains, Staphylococci, Salmonella and Streptococci. Antigens of infectious protozoan pathogens include, but are not limited to, antigens of Plasmodium, Trypanosoma, Leishmania and Toxoplasma. Further examples of antigens of pathogenic agents include antigens of fungal pathogens such as antigens of Cryptococcus neoformans, Histoplasma capsulatum, Coccidioides immitis, Blastomyces dermatitidis and Candida albicans.

Specific examples of tumor antigens which can be used according to the invention include, but not limited to 707-AP, AFP, ART-4, BAGE, beta-catenin/m, Bcr-abl, CAMEL, CAP-1, CASP-8, CDC27/m, CDK4/m, CEA, CT, Cyp-B, DAM, ELF2M, ETV6-AML1, G250, GAGE, GnT-V, Gp100, HAGE, HER-2/neu, HLA-A*0201-R1701, HPV-E7, HSP70-2M, HAST-2, hTERT (or hTRT), iCE, KIAA0205, LAGE, LDLR/FUT, MAGE, MART-1/Melan-A, MC1R, myosin/m, MUC1, MUM-1, -2, -3, NA88-A, NY-ESO-1, p190 minor bcr-abl, Pml/RAR.alpha., PRAME, PSA, PSM, RAGE, RU1 or RU2, SAGE, SART-1 or SART-3, TEL/AML1, TPI/m, TRP-1, TRP-2, TRP-2/INT2 and WT1.

Especially in the context of antigens included into the inventive polypeptides as L₁ and/or L₂, but also with respect to any protein-protein interaction such as receptor-ligand binding, it is possible to include a selection and/or evolutionary process for providing target binding-optimized sequences such as optimized antigens to exert an improved immune response thereto. A preferred process is ribosome display as outlined in detail in Schaffitzel et al. (2001) in: Protein-Protein Interactions, A Molecular Cloning Manual: In vitro selection and evolution of protein-ligand interaction by ribosome display (Golemis E., ed.), pages 535-567, Cold Spring Harbor Laboratory Press, New York. The ribosome display protocol has the advantage of being carried out completely in vitro at all steps of the selection process. Further possible selection processes are also known in the art and include phage display (Smith (1985) Science 228, 1315-1317; Winter et al. (1994) Annu. Rev. Immunol. 12, 433-455), yeast two-hybrid systems (Fields and Song (19899 Nature 340, 245-246; Chien et al. (1983) Proc. Natl. Acad. Sci. U.S.A. 88, 9578-9582), and cell surface display methods (Georgiu et al. (1993) Trends Biotechnol. 11, 6-10; Boder and Wittrup (1997) Nat. Biotechnol. 15, 553-557).

The ribosome display process can basically be used in two ways for optimization of antigens or other amino acid sequences involved in targeting a specific molecule by use of the polypeptides of the invention. Either, an antigen (or other binder) sequence can be selected first from an initial library of polypeptides sequences that can be as large as 10¹⁴ individual sequences, more typically 10⁹ to 10¹⁰ sequences, optionally employing evolutionary procedures as described in detail in Schaffitzel et al. (2001), supra. After selection of the optimized antigen sequences, the nucleotide sequence encoding it is cloned into an appropriate vector of the invention such that a polypeptide is expressed where the optimized antigen is included in or represents L₁ and/or L₂ according to formula (I) above.

According to an alternative embodiment of this aspect of the invention, a library of potential antigen encoding sequences is directly cloned into a nucleic acid of the invention such that each sequence encodes a polypeptide which is a part of or is, respectively, one or both of L₁ and L₂ as defined in formula (I), supra. The inventive polypeptides comprising an initial library of antigen sequences (or, in other embodiments, other binder sequences) are than expressed in vitro and selection of optimized antigen (or other binder) sequences is carried out according to the ribosome display methodology as outlined in detail in Schaffitzel et al. (2001), supra.

A further embodiment of polypeptides of the invention relates to polypeptides where L₁ and/or L₂ are or are coupled to, respectively, antibody sequences or parts of antibodys such as antibody fragments. In this context of the invention the term “antibody” is an immunoglobulin specifically binding to an antigen.

The term “antibody fragment” refers to a part of an antibody which retains the ability of the complete antibody to specifically bind to an antigen. Examples of antibody fragments include, but are not limited to, Fab fragments, Fab′ fragments, F(ab′)₂ fragments, heavy chain antibodys, single-domain antibodies (sdAb), scFv fragments, fragment variables (Fv), VH domains, VL domains, nanobodies, IgNARs (immunoglobulin new antigen receptors), di-scFv, bispecific T-cell engagers (BITEs), dual affinity re-targeting (DART) molecules, triple bodies, diabodis, a single-chain diabody and the like.

A “diabody” is a fusion protein or a bivalent antibody which can bind different antigens. A diabody is composed of two single protein chains (typically two scFv fragments) each comprising variable fragments of an antibody. Diabodies therefore comprise two antigen-binding sites and can, thus, target the same (monospecific diabody) or different antigens (bispecific diabody).

The term “single domain antibody” as used in the context of the present invention refers to antibody fragments consisting of a single, monomeric variable domain of an antibody. Simply, they only comprise the monomeric heavy chain variable regions of heavy chain antibodies produced by camelids or cartilaginous fish. Due to their different origins they are also referred to VHH or VNAR (variable new antigen receptor)-fragments. Alternatively, single-domain antibodies can be obtained by monomerization of variable domains of conventional mouse or human antibodies by the use of genetic engineering. They show a molecular mass of approximately 12-15 kDa and thus, are the smallest antibody fragments capable of antigen recognition. Further examples include nanobodies or nanoantibodies.

Antigen-binding entities useful in the context of the invention also include “antibody mimetic” which expression as used herein refers to compounds which specifically bind antigens similar to an antibody, but which compounds are structurally unrelated to antibodies. Usually, antibody mimetics are artificial peptides or proteins with a molar mass of about 3 to 20 kDa which comprise one, two or more exposed domains specifically binding to an antigen. Examples include inter alia the LACl-D1 (lipoprotein-associated coagulation inhibitor); affilins, e.g. human-γ B crystalline or human ubiquitin; cystatin; Sac7D from Sulfolobus acidocaldarius; lipocalin and anticalins derived from lipocalins; DARPins (designed ankyrin repeat domains); SH3 domain of Fyn; Kunits domain of protease inhibitors; monobodies, e.g. the 10thtype III domain of fibronectin; adnectins: knottins (cysteine knot miniproteins); atrimers; evibodies, e.g. CTLA4-based binders, affibodies, e.g. three-helix bundle from Z-domain of protein A from Staphylococcus aureus; Trans-bodies, e.g. human transferrin; tetranectins, e.g. monomeric or trimeric human C-type lectin domain; microbodies, e.g. trypsin-inhibitor-II, affilins; armadillo repeat proteins. Nucleic acids and small molecules are sometimes considered antibody mimetics as well (aptamers), but not artificial antibodies, antibody fragments and fusion proteins composed from these. Common advantages over antibodies are better solubility, tissue penetration, stability towards heat and enzymes, and comparatively low production costs.

As native penton base proteins do, the polypeptides of the invention assemble into pentameric complexes, 12 of which in turn assemble into virus-like particles (VLPs) in a buffer solution of preferably pH about 5.0 to about 8.0. Preferred examples are buffer conditions at or near physiological conditions such as PBS, pH 7.4, or TBS or TBS-T pH 7.2 to 7.6. Under such conditions, the polypeptides of the invention form VLPs at a temperature of about from about 20 to about 42° C. The present invention is also directed to such pentameric complexes and VLPs.

Further subject matter of the invention is a nucleic acid coding for the polypeptide as defined herein.

According to the present invention, the terms “nucleic acid” and “polynucleotide” are used interchangeably and refer to DNA, RNA or species containing one or more nucleotide analogues. Preferred nucleic acids or polynucleotides according to the present invention are DNA, most preferred double-stranded (ds) DNA. Nucleotide sequences of the present disclosure are shown from 5′ to 3′, and the IUPAC single letter code for bases is used, if not otherwise used as indicated.

Another embodiment relates to a nucleic acid prepared for insertion of the versatile segments L₁ and L₂ as defined in general formula (1). That is, this embodiment of the nucleic acid encodes segments A, B and C, but has insertion sites between the segments coding for A and B, and between the segments encoding B and C.

Thus, this embodiment can be represented by the following general formula (II):

5′-a-is₁-|₁-is ₂-b-is ₃-I₂-is ₄-c-3′ (II)

wherein
a is a nucleotide sequence encoding A of general formula (I);
b is a nucleotide sequence encoding B of general formula (I);
c is a nucleotide sequence encoding C of general formula (I); and
I₁, I₂ is each a nucleotide sequence;
is₁ to is₄ are each independently a nucleotide sequence comprising at least one insertion site.

An insertion site in the context of this embodiment of the invention is preferably a recognition sequence of a restriction enzyme or of a homing endonuclease. More preferably, the is1 to is4 are each different insertion sites, more particularly each is1 to is4 is a recognition sequence of different restriction enzymes. A preferred embodiment of the nucleic acid prepared for insertion of nucleotide sequences coding for L₁ and L₂ has a nucleotide sequence wherein is, comprises an EcoRI site, is₂ comprises a RsrII site, is₃ comprises a SacI site, and is₄ comprises a XbaI site.

Restriction enzyme sites are generally well-known to the skilled person. Preferred examples are as defined above, but restriction sites can be selected from a wide variety and guidance can be found at the various manufacturers of restriction enzymes such as New England Biolabs, Inc., Ipswich, Mass., USA.

Examples of such homing endonuclease (HE) sites include, but are not limited to, recognition sequences of PI-Scel, I-Ceul, I-Ppol, I-Hmul I-Crel, I-Dmol, PI-Pful and I-Msol, PI-Pspl, I-Scel, other LAGLIDAG group members and variants thereof, SegH and Hef or other GIY-YIG homing endonucleases, I-Apell, I-Anil, Cytochrome b mRNA maturase bl3, PI-TliI and PI-Tfull, PI-Thyl and others; see Stoddard B. L. (2005) Q. Rev. Biophys. 38, 49-95. Corresponding enzymes are commercially available, e. g. from New England Biolabs Inc., Ipswich, Mass., USA.

In preferred embodiments of the present invention, the above-defined nucleic acid additionally comprises at least one site for integration of the nucleic acid into a vector or host cell. The integration site may allow for a transient or genomic incorporation.

With respect to the integration into a vector, in particular into a plasmid or virus, the integration site is preferably compatible for integration of the nucleic acid into an adenovirus, adeno-associated virus (AAV), autonomous parvovirus, herpes simplex virus (HSV), retrovirus, rhadinovirus, Epstein-Barr virus, lentivirus, semliki forest virus or baculovirus.

Particularly preferred integration sites that may be incorporated into the nucleic acid of the present invention can be selected from the transposon element of Tn7, λ-integrase specific attachment sites and site-specific recombinases (SSRs), in particular LoxP site or FLP recombinase specific recombination (FRT) site. Further preferred mechanisms for integration of the nucleic acid according to the invention are specific homologous recombination sequences such as lef2-603/Orf1629.

In further preferred embodiments of the present invention, the nucleic acid as described herein additionally contains one or more resistance markers for selecting against otherwise toxic substances. Preferred examples of resistance markers useful in the context of the present invention include, but are not limited to, antibiotics such as ampicillin, chloramphenicol, gentamycin, spectinomycin, and kanamycin resistance markers.

The nucleic acid of the present invention may also contain one or more ribosome binding site(s) (RBS)

Further subject-matter of the present invention relates to a vector comprising a nucleic acid as defined above.

Preferred vectors of the present invention are plasmids, expression vectors, transfer vectors, more preferred eukaryotic gene transfer vectors, transient or viral vector-mediated gene transfer vectors. Other vectors according to the invention are viruses such as adenovirus vectors, adeno-associated virus (AAV) vectors, autonomous parvovirus vectors, herpes simples virus (HSV) vectors, retrovirus vectors, rhadinovirus vectors, Epstein-Barr virus vectors, lentivirus vectors, semliki forest virus vectors and baculovirus vectors.

Baculovirus vectors suitable for integrating a nucleic acid according to the invention (e.g. present on a suitable plasmid such as a transfer vector) are also subject matter of the present invention and preferably contain site-specific integration sites such as a Tn7 attachment site (which may be embedded in a lacZ gene for blue/white screening of productive integration) and/or a LoxP site. Further preferred baculovirus according to the invention contain (alternative to or in addition to the above-described integration sites) a gene for expressing a substance toxic for host flanked by sequences for homologous recombination. An example for a gene for expressing a toxic substance is the diphtheria toxin A gene. A preferred pair of sequences for homologous recombination is e.g. Isf2-603/Orf1629. The baculovirus can also contain further marker gene(s) as described above, including also fluorescent markers such as GFP, YFP and so on. Specific examples of corresponding baculovirus are, for example disclosed in WO 2010/100278 A1.

Further applicable vectors for use in the invention are disclosed in WO 2005/085456 A1.

Vectors useful in prokaryotic host cells comprise, preferably besides the above-exemplified marker genes (one or more thereof), an origin of replication (ori). Examples are BR322, ColE1, and conditional origins of replication such as OriV and R6Kγ, the latter being a preferred conditional origin of replication which makes the propagation of the vector of the present application dependent on the pir gene in a prokaryotic host. OriV makes the propagation of the vector of the present application dependent on the trfA gene in a prokaryotic host.

Furthermore, the present invention is directed to a host cell containing the nucleic acid of the invention and/or the vector of the present invention.

The host cells may be prokaryotic or eukaryotic. Eukaryotic host cells may for example be mammalian cells, preferably human cells. Examples of human host cells include, but are not limited to, HeLa, Huh7, HEK293, HepG2, KATO-III, IMR32,

MT-2, pancreatic β-cells, keratinocytes, bone-marrow fibroblasts, CHP212, primary neural cells, W12, SK-N-MC, Saos-2, WI38, primary hepatocytes, FLC4, 143TK, DLD-1, embryonic lung fibroblasts, primery foreskin fibroblasts, MRCS, and MG63 cells. Further preferred host cells of the present invention are porcine cells, preferably CPK, FS-13, PK-15 cells, bovine cells, preferably MDB, BT cells, bovine cells, such as FLL-YFT cells. Other eukaryotic cells useful in the context of the present invention are C. elegans cells. Further eukaryotic cells include yeast cells such as S. cerevisiae, S. pombe, C. albicans and P. pastoris. Furthermore, the present invention is directed to insect cells as host cells which include cells from S. frugiperda, more preferably Sf9, Sf21, Express Sf+, High Five H5 cells, and cells from D. melanogaster, particularly S2 Schneider cells. Further host cells include Dictyostelium discoideum cells and cells from parasites such as Leishmania spec.

Prokaryotic hosts according to the present invention include bacteria, in particular E. coli such as commercially available strains like TOP10, DH5α, HB101. BL21(DE3) etc.

The person skilled in the art is readily able to select appropriate vector construct/host cell pairs for appropriate propagation and/or transfer of the nucleic acid elements according to the present invention into a suitable host. Specific methods for introducing appropriate vector elements and vectors into appropriate host cells are equally known to the art and methods can be found in the latest edition of Ausubel et al. (ed.) Current Protocols In Molecular Biology, John Wiley & Sons, New York, USA.

In preferred embodiments of the present invention, the vector as defined above additionally comprises a site for site specific recombinases (SSRs), preferably one or more LoxP sites for Cre-lox specific recombination. In further preferred embodiments, the vector according to the present invention comprises a transposon element, preferably a Tn7 attachment site.

It is further preferred that the attachment site as defined above is located within a marker gene. This arrangement makes it feasible to select for successfully integrated sequences into the attachment site by transposition. According to preferred embodiments, such a marker gene is selected from luciferase, β-GAL, CAT, fluorescent encoding protein genes, preferably GFP, BFP, YFP, CFP and their variants, and the lacZα gene.

Furthermore, the present invention is directed to a host cell containing the nucleic acid of the invention and/or the vector of the present invention.

The host cells may be prokaryotic or eukaryotic. Eukaryotic host cells may for example be mammalian cells, preferably human cells. Examples of human host cells include, but are not limited to, HeLa, Huh7, HEK293, HepG2, KATO-III, IMR32,

MT-2, pancreatic β-cells, keratinocytes, bone-marrow fibroblasts, CHP212, primary neural cells, W12, SK-N-MC, Saos-2, WI38, primary hepatocytes, FLC4, 143TK, DLD-1, embryonic lung fibroblasts, primary foreskin fibroblasts, MRCS, and MG63 cells. Further preferred host cells of the present invention are porcine cells, preferably CPK, FS-13, PK-15 cells, bovine cells, preferably MDB, BT cells, bovine cells, such as FLL-YFT cells. Other eukaryotic cells useful in the context of the present invention are C. elegans cells. Further eukaryotic cells include yeast cells such as S. cerevisiae, S. pombe, C. albicans and P. pastoris. Furthermore, the present invention is directed to insect cells as host cells which include cells from S. frugiperda, more preferably Sf9, Sf21, Express Sf+, High Five H5 cells, and cells from D. melanogaster, particularly S2 Schneider cells. Further host cells include Dictyostelium discoideum cells and cells from parasites such as Leishmania spec.

Prokaryotic hosts according to the present invention include bacteria, in particular E. coli such as commercially available strains like TOP10, DH5α, HB101, BL21(DE3) etc.

The person skilled in the art is readily able to select appropriate vector construct/host cell pairs for appropriate propagation and/or transfer of the nucleic acid elements according to the present invention into a suitable host. Specific methods for introducing appropriate vector elements and vectors into appropriate host cells are equally known to the art and methods can be found in the latest edition of Ausubel et al. (ed.) Current Protocols In Molecular Biology, John Wiley & Sons, New York, USA.

The present invention also provides the polypeptide, the nucleic acid encoding such a polypeptide, the vector containing a polypeptide-encoding nucleic acid, the host cell comprising such a vector as well as the VLP as defined above for use as a medicament, in particular for use in the treatment and/or prevention of an infectious disease, an immune disease, tumour or cancer.

Therefore, the present invention is also directed to pharmaceutical compositions comprising a polypeptide as defined herein, a nucleic acid encoding such a polypeptide, a vector containing a polypeptide-encoding nucleic, a host cell comprising such a vector or a VLP as described above together with at least one pharmaceutically acceptable carrier, excipient and/or diluent.

Generally, the preparation of pharmaceutical compositions in the context of the present invention, their dosages and their routes of administration are known to the skilled person, and guidance can be found in the latest edition of Remington's Pharmaceutical Sciences (Mack publishing Co., Eastern, Pa., USA).

The pharmaceutical compositions of the invention contain a therapeutically effective amount of the active ingredient as outlined above. The therapeutically effective amount depends on the active ingredient and in particular on the route of administration. The pharmaceutical composition according to the invention will preferably be applied by parenteral administration, in particular by infusion such as intravenous, intraarterial or intraosseous infusion, or by injection, e.g. intravenous, intraarterial, intraperitoneal, intramuscular, intradermal, subcutaneous or intrathecal injection. In the case of anti-tumor therapy, the pharmaceutical composition such as a pharmaceutical composition containing VLPs according to the invention, can also be administered by intra-tumoral injection.

Inventive solutions for injection or infusion typically contain VLPs of the invention in water or an aqueous buffer solution, preferably an isotonic buffer at physiological pH. Liquid pharmaceutical compositions of the invention may contain further ingredients such as pharmaceutically acceptable stabilizers, suspending aids, emulsifyers and the likes. Further ingredients of the pharmaceutical composition of the invention are adjuvants, in particular in the context of application of the constructs of the invention for vaccination purposes.

Further subject matter of the invention are methods of treatment making use of the beneficial properties of the polypeptides, nucleic acids, host cells, vectors and/or VLPs of the invention. In a preferred embodiment, the invention provides a method for the prevention and/or treatment of an infectious disease comprising the step of administering to a subject, preferably a human, a therapeutically effective amount of the pharmaceutical composition as defined above, wherein the pharmaceutical composition comprises VLPs of the invention containing antigens (particularly comprised in L₁ and/or L₂ of the inventive polypeptide as defined above) of the infective agent causing the infectious disease. Another embodiment is a method for preventing and/or treating a tumor or cancer disease the step of administering a therapeutically effective amount of the pharmaceutical composition as defined above to a subject, preferably a human, wherein the pharmaceutical composition comprises VLPs of the invention containing one or more tumour antigens (particularly comprised in L₁ and/or L₂ of the inventive polypeptide as defined above).

The present invention is further directed to a method for producing the polypeptide as described herein comprising the step of cultivating the recombinant host cell in a suitable medium, wherein the host cell comprises a vector which comprises a nucleic encoding the polypeptide, under conditions allowing the expression of said polypeptide.

Preferably, the method for producing the polypeptide of the invention further comprises the step of recovering the expressed polypeptide from the host cells and/or the medium. Even more preferred, the method also comprises the step of purifying the recovered polypeptide by purification means known in the art such as centrifugation, gel chromatography, affinity chromatography etc.

The invention also provides a method for producing the VLP as defined herein comprising the step of incubating a solution of the polypeptide under conditions allowing the assembly of the polypeptide into a VLP as outlined before. The proper formation of VLPs can be tested by inspecting a sample solution with an electron microscope.

### BRIEF DESCRIPTION OF THE DRAWINGS

FIG. 1A is an image of a dodecahedron formed by 60 copies of base proteins of certain Adenovirus serotypes. FIG. 1B is an image of a adenovirus base protein which can be split into a crown domain and a multimerization domain. Within the multimerization domain, the termini generated by splitting can be reconnected by short oligopeptide linkers yielding contiguous polypeptide chains. Both he N and C-termini of the base protein are contained in the multimerization domain.

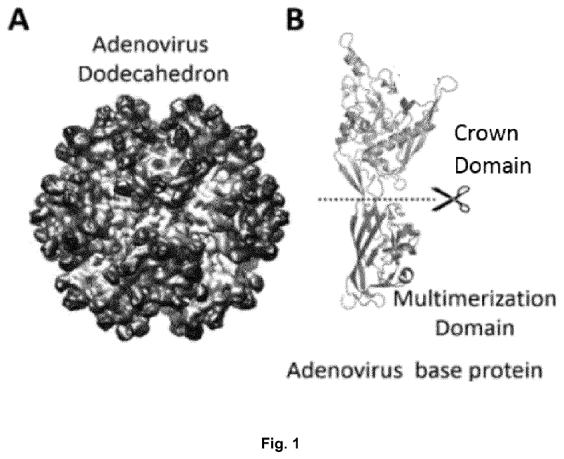

FIG. 2 is a schematical overview of the jellyroll fold domain of a preferred embodiment of the inventive polypeptide based on the penton base protomer of hAd3.

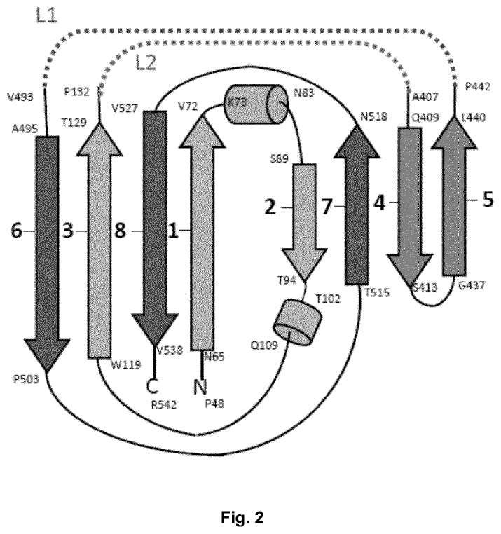

FIG. 3 is a schematic view of an adenovirus base proteinshowing positions of the crown domain and the multimerization domain. The N and C-termini of the base protein are contained in the multimerization domain, which mediates penton and dodecahedron formation. Removal of the crown domain yields an autonomous multimerization domain which, in place of formerly the crown domain, now contains oligopeptides, polypeptides, proteins or protein complexes as shown schematically towards from middle to right. The multimerization domains now give rise to virus-like particles presenting these entities on their surface. Naturally, crown domains and engineered crown domains derived from a range of Adenovirus serotypes can also be fitted on out multimerization domain scaffold, including engineered crown domains containing heterologous peptide and polypeptide sequences.

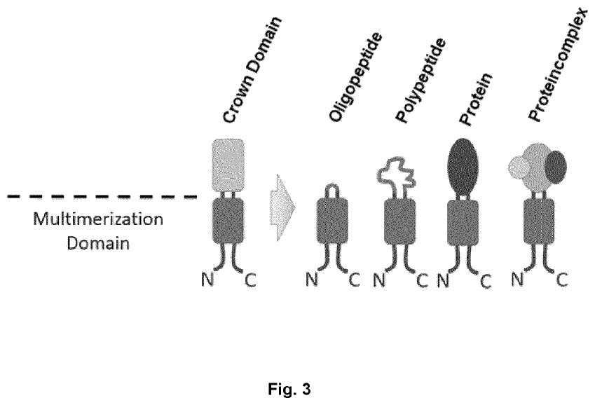

FIG. 4 is a schematic view of an Adenovirus derived dodecahedral display platform (ADDomer). The original particle is shown on the left. The base protein is formed by the multimerization domain and the crown domain. 60 base proteins form a virus-like particle (VLP). ADDomers displaying (instead of the crown domain) multiple copies of oligopeptides, polypeptides and protein domains, proteins or protein complexes are shown on the right.

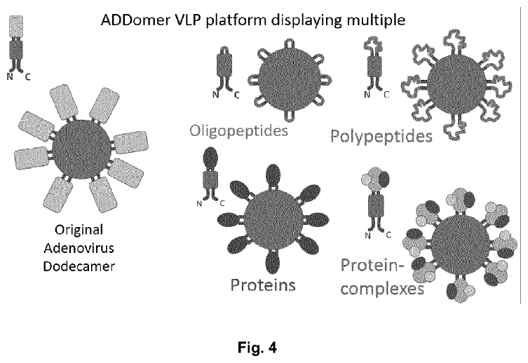

FIG. 5 is a schematic representation of vector pACEBac_VAJB-CHIK.

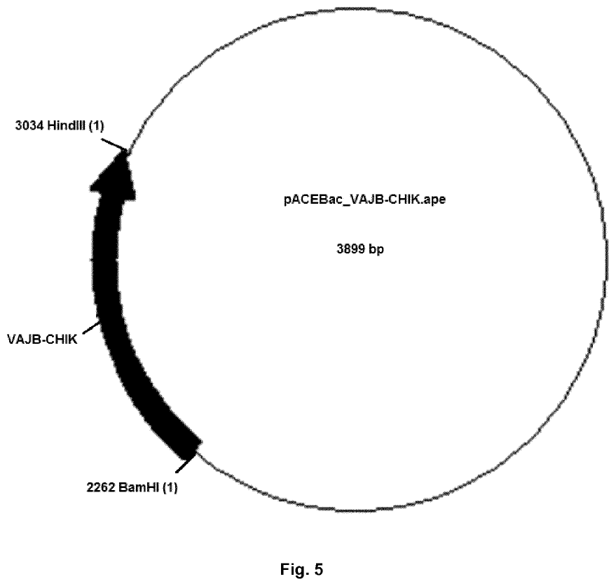

### DETAILED DESCRIPTION OF THE INVENTION

The present invention is further illustrated by the following non-limiting example:

### Example

A nucleic acid with the sequence denoted DNAsegVAJB-CHIK (SEQ ID NO: 26; flanked by BamHI site at the 5′ end and by a HindIII site at the 3′ end; see underlined sequence below) was synthesized by a commercial supplier:

ggatccatgaggagacgagccgtgctaggcggagcggtggtgtatccgga
gggtcctcctccttcttacgagagcgtgatgcagcaacaggcggcgatga
tacagcccccactggaggctcccttcgtacccccacggtacctggcgcct
acggaagggagaaacagcattcgttactcggagctgtcgcccctgtacga
taccaccaagttgtatctggtggacaacaagtcggcggacatcgcctccc
tgaactatcagaacgaccacagcaacttcctgaccacggtggtgcagaac
aatgactttacccccacggaggctagcacccagaccatcaactttgacga
gcggtcgcgatggggcggtcagctgaagaccatcatgcacaccaacatgc
ccggaggtgaaaacctgtattttcagagcaccaaagataactttaacgtg
tataaagcgacccgcccgtatctggcgcatggaggtgcagagcaggtcta
ctggtcgctccctgacatgatgcaagacccagtcaccttccgctccacaa
gacaagtcaacaactacccagtggtgggtgcagagcttatgcccggtgga
agcggaggtagcgttcctgctctcacagatcacgggaccctgccgttacg
cagcagtatccggggagtccagcgcgtgaccgttactgacgccagacgcc
gcacctgtccctacgtttacaaggccctgggcatagtcgcgccgcgcgtt
ctttcaagccgcactttctgataagctt

The construct was cloned into transfer plasmid pACEBac (Geneva Biotech, Geneva, Switzerland) using cleavage sites BamHI and HindIII, giving rise to the construct pACEBac_VAJB-CHIK (SEQ ID NO: 27):

accggttgacttgggtcaactgtcagaccaagtttactcatatatacttt
agattgatttaaaacttcatttttaatttaaaaggatctaggtgaagatc
ctttttgataatctcatgaccaaaatcccttaacgtgagttttcgttcca
ctgagcgtcagaccccgtagaaaagatcaaaggatcttcttgagatcctt
tttttctgcgcgtaatctgctgcttgcaaacaaaaaaaccaccgctacca
gcggtggtttgtttgccggatcaagagctaccaactctttttccgaaggt
aactggcttcagcagagcgcagataccaaatactgttcttctagtgtagc
cgtagttaggccaccacttcaagaactctgtagcaccgcctacatacctc
gctctgctaatcctgttaccagtggctgctgccagtggcgataagtcgtg
tcttaccgggttggactcaagacgatagttaccggataaggcgcagcggt
cgggctgaacggggggttcgtgcacacagcccagcttggagcgaacgacc
tacaccgaactgagatacctacagcgtgagctatgagaaagcgccacgct
tcccgaagggagaaaggcggacaggtatccggtaagcggcagggtcggaa
caggagagcgcacgagggagcttccagggggaaacgcctggtatctttat
agtcctgtcgggtttcgccacctctgacttgagcgtcgatttttgtgatg
ctcgtcaggggggcggagcctatggaaaaacgccagcaacgcggcctttt
tacggttcctggccttttgctggccttttgctcacatgttctttcctgcg
ttatcccctgattgacttgggtcgctcttcctgtggatgcgcagatgccc
tgcgtaagcgggtgtgggcggacaataaagtcttaaactgaacaaaatag
atctaaactatgacaataaagtcttaaactagacagaatagttgtaaact
gaaatcagtccagttatgctgtgaaaaagcatactggacttttgttatgg
ctaaagcaaactcttcattttctgaagtgcaaattgcccgtcgtattaaa
gaggggcgtggccaagggcatgtaaagactatattcgcggcgttgtgaca
atttaccgaacaactccgcggccgggaagccgatctcggcttgaacgaat
tgttaggtggcggtacttgggtcgatatcaaagtgcatcacttcttcccg
tatgcccaactttgtatagagagccactgcgggatcgtcaccgtaatctg
cttgcacgtagatcacataagcaccaagcgcgttggcctcatgcttgagg
agattgatgagcgcggtggcaatgccctgcctccggtgctcgccggagac
tgcgagatcatagatatagatctcactacgcggctgctcaaacttgggca
gaacgtaagccgcgagagcgccaacaaccgcttcttggtcgaaggcagca
agcgcgatgaatgtcttactacggagcaagttcccgaggtaatcggagtc
cggctgatgttgggagtaggtggctacgtctccgaactcacgaccgaaaa
gatcaagagcagcccgcatggatttgacttggtcagggccgagcctacat
gtgcgaatgatgcccatacttgagccacctaactttgttttagggcgact
gccctgctgcgtaacatcgttgctgctgcgtaacatcgttgctgctccat
aacatcaaacatcgacccacggcgtaacgcgcttgctgcttggatgcccg
aggcatagactgtacaaaaaaacagtcataacaagccatgaaaaccgcca
ctgcgccgttaccaccgctgcgttcggtcaaggttctggaccagttgcgt
gagcgcatacgctacttgcattacagtttacgaaccgaacaggcttatgt
caactgggttcgtgccttcatccgtttccacggtgtgcgtcacccggcaa
ccttgggcagcagcgaagtcgccataacttcgtatagcatacattatacg
aagttatctgtaactataacggtcctaaggtagcgagtttaaacactagt
atcgattcgcgacctactccggaatattaatagatcatggagataattaa
aatgataaccatctcgcaaataaataagtattttactgttttcgtaacag
ttttgtaataaaaaaacctataaatattccggattattcataccgtccca
ccatcgggcgcggatccAtgaggagacgagccgtgctaggcggagcggtg
gtgtatccggagggtcctcctccttcttacgagagcgtgatgcagcaaca
ggcggcgatgatacagcccccactggaggctcccttcgtacccccacggt
acctggcgcctacggaagggagaaacagcattcgttactcggagctgtcg
cccctgtacgataccaccaagttgtatctggtggacaacaagtcggcgga
catcgcctccctgaactatcagaacgaccacagcaacttcctgaccacgg
tggtgcagaacaatgactttacccccacggaggctagcacccagaccatc
aactttgacgagcggtcgcgatggggcggtcagctgaagaccatcatgca
caccaacatgcccGGAGGTgaaaacctgtattttcagagcaccaaagata
actttaacgtgtataaagcgacccgcccgTatctggcgcatGGAGGTGca
gagcaggtctactggtcgctccctgacatgatgcaagacccagtcacctt
ccgctccacaagacaagtcaacaactacccagtggtgggtgcagagctta
tgcccGGTGGAagcggAggtagcgttcctgctctcacagatcacgggacc
ctgccgttacgcagcagtatccggggagtccagcgcgtgaccgttactga
cgccagacgccgcacctgtccctacgtttacaaggccctgggcatagtcg
cgccgcgcgttctttcaagccgcactttctgataagcttccatcaacttt
gacgagcggtcgcgatggggcggtcagctgaagaccatcatgcacaccaa
catgcccaacgtgaacgagtacatgttcagcaacaagttcaaggcgaggg
agcttgtcgagaagtactagaggatcataatcagccataccacatttgta
gaggttttacttgctttaaaaaacctcccacacctccccctgaacctgaa
acataaaatgaatgcaattgttgttgttaacttgtttattgcagcttata
atggttacaaataaagcaatagcatcacaaatttcacaaataaagcattt
ttttcactgcattctagttgtggtttgtccaaactcatcaatgtatctta
tcatgtctggatctgatcactgcttgagcctagaagatccggctgctaac
aaagcccgaaaggaagctgagttggctgctgccaccgctgagcaataact
atcataacccctagggtatacccatctaattggaaccagataagtgaaat
ctagttccaaactattttgtcatttttaattttcgtattagcttacgacg
ctacacccagttcccatctattttgtcactcttccctaaataatccttaa
aaactccatttccacccctcccagttcccaactattttgtccgcccaca

DNA sequencing was used to verify the proper insertion. The open reading frame encodes the protein VAJB-CHIK (SEQ ID NO: 28) which contains the major neutralizing epitope from Chikungunya virus STKDNFNVYKATRPYLAH (SEQ ID NO: 29) in loop L1.

VAJB-CHIK (SEQ ID NO: 28):
MRRRAVLGGA VVYPEGPPPS YESVMQQQAA MIQPPLEAPF
VPPRYLAPTE GRNSIRYSEL SPLYDTTKLY LVDNKSADIA
SLNYQNDHSN FLTTVVQNND FTPTEASTQT INFDERSRWG
GQLKTIMHTN MPGGENLYFQ STKDNFNVYK ATRPYLAHGG
AEQVYWSLPD MMQDPVTFRS TRQVNNYPVV GAELMPGGSG
GSVPALTDHG TLPLRSSIRG VQRVIVTDAR RRTCPYVYKA
LGIVAPRVLS SRTF

pACEBac_VAJB-CHIK was then used to transform DH10EMBacY cells (Geneva Biotech, Geneva, Switzerland) harbouring the baculoviral genome EMBacY as an artificial chromosome (described in Fitzgerald D J et al. Nat Methods. 2006 Dec. 3(12):1021-32 PMID: 17117155). Composite baculovirus with the expression cassette for VAJB1 integrated by Tn7 transposition in the DH10EMBacY cells was then identified by blue/white screening, and recombinant baculovirus generated as described (ibid). Spodoptera frugiperda line 21 (Sf21) insect cell cultures were infected with baculovirus thus generated as described by Fitzgerald et al. (2006) Nat Methods, supra.

Large-scale (100 ml-500 ml) expression was carried out in Trichoplusia ni Hi5 cells in shaker flasks and recombinant protein expression followed by measuring yellow fluorescent protein (YFP) fluorescence as described (Fitzgerald D J et Nat Methods. 2006 Dec. 3(12):1021-32 PMID: 17117155). When YFP fluorescence reached a plateau (normally after 72 hours after proliferation arrest in the cell culture, see Fitzgerald et al, Nat Methods 2006), insect cell cultures were harvested and cells pelleted by centrifugation (4000 g, 10 min). Cells were frozen in liquid nitrogen and stored at −80 degrees Celsius.or protein preparation, cells were lysed by freeze-thawing in phosphate buffered saline (PBS) containing whole protease inhibitor cocktail (Roche Ltd). Protein was purified by loading on a sucrose gradient from 15% to 40% w/v sucrose and ultracentrifugation overnight at 100.000 g. The gradient was harvested and protein content identified by means of denaturing polyacrylamide gel electrophoresis (SDS-PAGE) followed by Commassie Brilliant Blue staining. The fractions containing VAJB1 were pooled and dialysed against PBS (or HEPES 10 mM, pH 7.4, 50 mM NaCl). A second purification step was performed on 5 ml HiQ column (BioRAD) using a linear gradient from 50 mM to 500 mM NaCl. Pentamer and Dodecamer formation was verified by negative stain (uranyl acetate) electron microscopy.

1. A polypeptide having the structure of formula (I)
A-L₁-B-L₂-C (I)
wherein
A is an N-terminal amino acid stretch of an adenovirus penton base protein;
B is an amino acid stretch of an adenovirus penton base protein;
C is a C-terminal amino acid stretch of an adenovirus penton base;
wherein B is an amino acid stretch located between A and C in the sequence of said adenovirus penton base;
wherein A, B and C form the jellyroll fold domain of said adenovirus penton base protein;
wherein L₁ and L₂ are independently from one another selected from the group consisting of an oligopeptide, a polypeptide, a protein, and a protein complex;
and
wherein said oligopeptide, polypeptide, protein and protein complex, respectively, are either essentially non-adenoviral or, if adenoviral, are from an adenovirus having a different serotype compared to the serotype of the adenovirus from which said amino acid stretches A, B and C are derived.

2. The polypeptide of claim 1, wherein amino acid stretch A comprises beta sheets 1, 2 and 3 of the jellyroll fold domain of said adenovirus.

3. The polypeptide of claim 1, wherein amino acid stretch B comprises beta sheets 4 and 5 of the jellyroll fold domain of said adenovirus.

4. The polypeptide according to claim 1, wherein amino acid stretch C comprises beta sheets 6, 7 and 8 of the jellyroll fold domain of adenovirus.

5. The polypeptide according to claim 1, wherein amino acid stretches A, B, and C have an amino acid sequence each independently selected from the group consisting of penton bases of human adenovirus serotype 2, human adenovirus serotype 3, human adenovirus serotype 4, human adenovirus serotype 5, human adenovirus serotype 7, human adenovirus serotype 11, human adenovirus serotype 12, human adenovirus serotype 17, human adenovirus serotype 25, human adenovirus serotype 35, human adenovirus serotype 37, human adenovirus serotype 41, gorilla adenovirus, chimpanzee adenovirus, simian adenovirus serotype 18, simian adenovirus serotype 20, simian adenovirus serotype 49, rhesus adenovirus serotype 51, rhesus adenovirus serotype 52, and rhesus adenovirus serotype 53.

6. The polypeptide of claim 5, wherein amino acid stretch A has the following consensus sequence (SEQ ID NO: 21):
(U)₁₋₄₇PTX₁GRNSIRYSX₂X₃x₄PX₅X₆DTTX₇X₃YLVDNKSADIASL
NYQNDHSNFX₅TTVX₉QNNDX₁₀X₁₁PX₁₂EAX₁₃TQTINX₁₄DX₁₅RSR
WGX₁₆X₁₇LKTIX₁₈X₁₉TZ₁Z₂Z₃Z₄Z₅Z₆Z₇Z₈Z₉Z₁₀Z₁₁Z₁₂Z₁₃
Z₁₄Z₁₅
wherein: amino acid stretch A ends on the C-terminal side before Z₁ at residue T or at an amino acid from Z₁ to Z₁₅
U is any or no amino acid
X₁ is E or G
X₂ is E or S
X₃ is L or V
X₄ is A or S
X₅ is L or Q
X₆ is Y or E
X₇ is R or K
X₈ is V or L
X₉ is V or I
X₁₀ is F or Y
X₁₁ is T or S
X₁₂ is A or T or I or G
X₁₃ is S or G
X₁₄ is F or L
X₁₅ is E or D
X₁₆ is A or G
X₁₇ is D or Q
X₁₈ is L or M
X₁₉ is H or R
Z₁, if present, is N
Z₂, if present, is M
Z₃, if present, is P
Z₄, if present, is N
Z₅, if present, is V or I
Z₆, if present, is N
Z₇, if present, is E or D
Z₈, if present, is Y or F
Z₉, if present, is M
Z₁₀, if present, is F or S or Y
Z₁₁, if present, is T or S
Z₁₂, if present, is S or N
Z₁₃, if present, is K
Z₁₄, if present, is F
Z₁₆, if present, is K

7. The polypeptide of claim 6, wherein amino acid stretch A is selected from the group consisting of the following sequences:
from an amino acid selected from positions 1 to 48, to an amino acid selected from positions 129 to 144 of UniProt Acc. No. Q2Y0H9;
from an amino acid selected from positions 1 to 48, to an amino acid selected from positions 129 to 144 of UniProt Acc. No. P03276;
from an amino acid selected from positions 1 to 44, to an amino acid selected from positions 125 to 140 of UniProt Acc. No. Q2KSF3;
from an amino acid selected from positions 1 to 48, to an amino acid selected from positions 129 to 144 of UniProt Acc. No. P12538;
from an amino acid selected from positions 1 to 48, to an amino acid selected from positions 129 to 144 of UniProt Acc. No. Q9JFT6;
from an amino acid selected from positions 1 to 48, to an amino acid selected from positions 129 to 144 of UniProt Acc. No. D2DM93;
from an amino acid selected from positions 1 to 38, to an amino acid selected from positions 119 to 134 of UniProt Acc. No. P36716;
from an amino acid selected from positions 1 to 35, to an amino acid selected from positions 116 to 131 of UniProt Acc. No. F1DT65;
from an amino acid selected from positions 1 to 43, to an amino acid selected from positions 124 to 139 of UniProt Acc. No. M0QUK0;
from an amino acid selected from positions 1 to 49, to an amino acid selected from positions 130 to 145 of UniProt Acc. No. Q7T941;
from an amino acid selected from positions 1 to 35, to an amino acid selected from positions 116 to 131 of UniProt Acc. No. Q912J1;
from an amino acid selected from positions 1 to 46, to an amino acid selected from positions 127 to 142 of UniProt Acc. No. F8WQN4;
from an amino acid selected from positions 1 to 49, to an amino acid selected from positions 130 to 145 of UniProt Acc. No. E5L3Q9;
from an amino acid selected from positions 1 to 44, to an amino acid selected from positions 125 to 140 of UniProt Acc. No. G9G849;
from an amino acid selected from positions 1 to 46, to an amino acid selected from positions 127 to 142 of UniProt Acc. No. H8PFZ9;
from an amino acid selected from positions 1 to 45, to an amino acid selected from positions 126 to 141 of UniProt Acc. No. F6KSU4;
from an amino acid selected from positions 1 to 46, to an amino acid selected from positions 127 to 142 of UniProt Acc. No. F2WTK5;
from an amino acid selected from positions 1 to 43, to an amino acid selected from positions 124 to 139 of UniProt Acc. No. A0A0A1EWW1;
from an amino acid selected from positions 1 to 43, to an amino acid selected from positions 124 to 139 of UniProt Acc. No. A0A0A1EWX7; and
from an amino acid selected from positions 1 to 44, to an amino acid selected from positions 125 to 140 of UniProt Acc. No. A0A0A1EWZ7.

8. The polypeptide according to claim 5, wherein amino acid stretch B of above general formula (I) has the following sequence (SEQ ID NO: 22):
Z₁₇Z₁₈Z₁₉Z₂₀Z₂₁Z₂₂Z₂₃Z₂₄Z₂₅Z₂₆Z₂₇QVYWSLPDX₂₀MX₂₁DP
VTFRSTX₂₂QX₂₃X₂₄NX₂₅PVVGX₂₆ELZ₂₈Z₂₉Z₃₀
wherein: amino acid stretch B begins on the N-terminal side at an amino acid from Z₁₇ to Z₂₇ or at amino acid Q after Z₂₇;
amino acid stretch B ends on the C-terminal side before Z₂₈ at amino acid L or at an amino acid from Z₂₈ to Z₃₀,
Z₁₇, if present, is L or S
Z₁₈, if present, is T or P or C
Z₁₉, if present, is T or P
Z₂₀, if present, is P or S or A or R
Z₂₁, if present, is N or D
Z₂₂, if present, is G or V
Z₂₃, if present, is H or T
Z₂₄, if present, is C
Z₂₅, if present, is G
Z₂₆, if present, is A or V or S
Z₂₇, if present, is E or Q
X₂₀ is L or M
X₂₁ is Q or K
X₂₂ is Q or R or S
X₂₃ is V or I
X₂₄ is S or N
X₂₅ is Y or F
X₂₆ is A or V
Z₂₈, if present, is M or L
Z₂₉, if present, is P
Z₃₀, if present, is V or F

9. The polypeptide of claim 8, wherein amino acid stretch B is selected from the group consisting of the following sequences:
from an amino acid selected from positions 398 to 409, to an amino acid selected from positions 440 to 443 of UniProt Acc. No. Q2Y0H9;
from an amino acid selected from positions 425 to 436, to an amino acid selected from positions 467 to 470 of UniProt Acc. No. P03276;
from an amino acid selected from positions 379 to 390, to an amino acid selected from positions 421 to 444 of UniProt Acc. No. Q2KSF3;
from an amino acid selected from positions 425 to 436, to an amino acid selected from positions 467 to 470 of UniProt Acc. No. P12538;
from an amino acid selected from positions 398 to 409, to an amino acid selected from positions 440 to 443 of UniProt Acc. No. Q9JFT6;
from an amino acid selected from positions 415 to 426, to an amino acid selected from positions 457 to 460 of UniProt Acc. No. D2DM93;
from an amino acid selected from positions 351 to 362, to an amino acid selected from positions 393 to 397 of UniProt Acc. No. P36716;
from an amino acid selected from positions 370 to 381, to an amino acid selected from positions 413 to 416 of UniProt Acc. No. F1DT65;
from an amino acid selected from positions 388 to 399, to an amino acid selected from positions 440 to 443 of UniProt Acc. No. M0QUK0;
from an amino acid selected from positions 445 to 456, to an amino acid selected from positions 497 to 500 of UniProt Acc. No. Q7T941;
from an amino acid selected from positions 372 to 383, to an amino acid selected from positions 414 to 417 of UniProt Acc. No. Q912J1;
from an amino acid selected from positions 362 to 373, to an amino acid selected from positions 404 to 407 of [Please insert UniProt Acc. No. F8WQN4;
from an amino acid selected from positions 416 to 427, to an amino acid selected from positions 458 to 461 of UniProt Acc. No. E5L3Q9;
from an amino acid selected from positions 372 to 383, to an amino acid selected from positions 420 to 423 of UniProt Acc. No. G9G849;
from an amino acid selected from positions 353 to 364, to an amino acid selected from positions 395 to 398 of UniProt Acc. No. H8PFZ9;
from an amino acid selected from positions 358 to 369, to an amino acid selected from positions 400 to 403 of UniProt Acc. No. F6KSU4;
from an amino acid selected from positions 356 to 367, to an amino acid selected from positions 398 to 401 of UniProt Acc. No. F2WTK5;
from an amino acid selected from positions 352 to 363, to an amino acid selected from positions 394 to 397 of UniProt Acc. No. A0A0A1EWW1;
from an amino acid selected from positions 350 to 361, to an amino acid selected from positions 392 to 395 of UniProt Acc. No. A0A0A1EWX7; and
from an amino acid selected from positions 351 to 362, to an amino acid selected from positions 393 to 396 of UniProt Acc. No. A0A0A1EWZ7.

10. The polypeptide according to claim 5, wherein amino acid stretch C of above general formula (I) has the following sequence (SEQ ID NO: 23):
Z₃₂Z₃₂Z₃₃ALTDHGTLPLRSSIX₂₇GVQRVTX₂₈TDARRRTCPYVYKA
LGIVX₃₀PX₃₁VLSSRTF
wherein: amino acid stretch C begins on the N-terminal side at an amino acid from Z₃₁ to Z₃₃ or at amino acid A after Z₃₃;
Z₃₁, if present, is N
Z₃₂, if present, is V
Z₃₃, if present, is P
X₂₇ is R or S or G
X₂₈ is V or I
X₂₉ is Y or H
X₃₀ is A or S
X₃₁ is R or K

11. The polypeptide of claim 10, wherein the amino acid stretch C is selected from the group consisting of the following sequences:
from an amino acid selected from positions 492 to 495, to the C-terminal amino acid of UniProt Acc. No. Q2Y0H9;
from an amino acid selected from positions 519 to 522, to the C-terminal amino acid of UniProt Acc. No. P03276;
from an amino acid selected from positions 466 to 469, to the C-terminal amino acid of UniProt Acc. No. Q2KSF3;
from an amino acid selected from positions 492 to 495, to the C-terminal amino acid of UniProt Acc. No. P12538;
from an amino acid selected from positions 465 to 468, to the C-terminal amino acid of UniProt Acc. No. Q9JFT6;
from an amino acid selected from positions 482 to 485, to the C-terminal amino acid of UniProt Acc. No. D2DM93;
from an amino acid selected from positions 419 to 422, to the C-terminal amino acid of UniProt Acc. No. P36716;
from an amino acid selected from positions 438 to 441, to the C-terminal amino acid of [Please insert UniProt Acc. No. F1DT65;
from an amino acid selected from positions 455 to 458, to the C-terminal amino acid of UniProt Acc. No. M0QUK0;
from an amino acid selected from positions 522 to 525, to the C-terminal amino acid of UniProt Acc. No. Q7T941;
from an amino acid selected from positions 439 to 442, to the C-terminal amino acid of UniProt Acc. No. Q912J1;
from an amino acid selected from positions 429 to 432, to the C-terminal amino acid of UniProt Acc. No. F8WQN4;
from an amino acid selected from positions 483 to 486, to the C-terminal amino acid of UniProt Acc. No. E5L3Q9;
from an amino acid selected from positions 445 to 448, to the C-terminal amino acid of UniProt Acc. No. G9G849;
from an amino acid selected from positions 420 to 423, to the C-terminal amino acid of UniProt Acc. No. H8PFZ9;
from an amino acid selected from positions 425 to 428, to the C-terminal amino acid of UniProt Acc. No. F6KSU4;
from an amino acid selected from positions 423 to 426, to the C-terminal amino acid of UniProt Acc. No. F2WTK5;
from an amino acid selected from positions 419 to 422, to the C-terminal amino acid of UniProt Acc. No. A0A0A1EWW1;
from an amino acid selected from positions 417 to 420, to the C-terminal amino acid of UniProt Acc. No. A0A0A1EWX7; and
from an amino acid selected from positions 418 to 421, to the C-terminal amino acid of UniProt Acc. No. A0A0A1EWZ7.

12. The polypeptide according to claim 5 wherein amino acid stretch A has the sequence of positions 1 to 132 of UniProt Acc. No. Q2Y0H9, amino acid stretch B has the sequence of positions 407 to 442 of UniProt Acc. No. Q2Y0H9, and amino acid stretch C has the sequence of positions 493 to 544 of UniProt Acc. No. Q2Y0H9.

13. The polypeptide according to claim 1, wherein L₁ and/or L₂ is an oligopeptide having a sequence of 4 to 40 amino acids being selected from amino acids G and S.

14. The polypeptide of claim 13 wherein L₁ and/or L₂ is independently selected from the group consisting of GGGS (SEQ ID NO: 24) and GGSGGS (SEQ ID NO: 25).

15. The polypeptide according to clam 1, wherein L₁ is an adenoviral sequence comprising an RGD loop of an adenovirus penton base having a different serotype compared to the serotype of the adenovirus(es) from which said amino acid stretches A, B and C are derived and/or L₂ is an adenoviral sequence comprising a variable loop of an adenovirus penton base having a different serotype compared to the serotype of the adenovirus from which said amino acid stretches A, B and C are derived.

16. The polypeptide of claim 15 wherein L₁ is a big fragment of a crown domain of an adenovirus penton base having a different serotype compared to the serotype of the adenovirus(es) from which said amino acid stretches A, B and C are derived and L₂ is a small fragment of a crown domain of an adenovirus penton base having a different serotype compared to the serotype of the adenovirus(es) from which said amino acid stretches A, B and C are derived.

17. The polypeptide of claim 16 wherein the big and the small fragment are derived from a penton base protein of the same adenovirus.

18. The polypeptide of claim 16 wherein the big and the small fragment are derived from a penton base protein of different adenoviruses.

19. The polypeptide according to claim 15 wherein one or more non-adenoviral sequences are inserted into the RGD loop and/or variable loop.

20. The polypeptide according to claim 1 wherein L₁ and/or L₂ comprise an antigen.

21. The polypeptide of claim 20 wherein the antigen is selected from the group consisting of an antigen of an infectious agent and a tumour antigen.

22. The polypeptide according to any claim 1 wherein a nucleic acid, a drug, label and/or binding partner of a biological binding pair is/are coupled to L₁ and/or L₂.

23. (canceled)

24. A nucleic acid comprising a sequence having the following general formula (II)
5′-a-is₁-I₁-is₂-b-is ₃-I₂-is₄-c-3′ (II)
wherein
a is a nucleotide sequence encoding A of general formula (I);
b is a nucleotide sequence encoding B of general formula (I);
c is a nucleotide sequence encoding C of general formula (I);
I₁, I₂ are each a nucleotide sequence; and
is₁ to is₄ are each independently a nucleotide sequence comprising at least one insertion site; and
wherein general formula (I) is
A-L₁-B-L₂-C (I)
wherein
A is an N-terminal amino acid stretch of an adenovirus penton base protein;
B is an amino acid stretch of an adenovirus penton base protein;
C is a C-terminal amino acid stretch of an adenovirus penton base;
wherein B is an amino acid stretch located between A and C in the sequence of said adenovirus penton base;
wherein A, B and C form the jellyroll fold domain of said adenovirus penton base protein;
wherein L₁ and L₂ are independently from one another selected from the group consisting of an oligopeptide, a polypeptide, a protein, and a protein complex;
and
wherein said oligopeptide, polypeptide, protein and protein complex, respectively, are either essentially non-adenoviral or, if adenoviral, are from an adenovirus having a different serotype compared to the serotype of the adenovirus from which said amino acid stretches A, B and C are derived.

25. The nucleic acid of claim 24 wherein is₁ to is₄ are selected from the group consisting of recognition sequences of restriction enzymes, and recognition sequences of homing endonucleases.

26. The nucleic acid of claim 25 wherein is₁ comprises an EcoRI site, is₂ comprises a RsrII site, is₃ comprises a SacI site, and is₄ comprises a XbaI site.

27-36. (canceled)

37. The polypeptide of claim 1 provided in a pentameric complex or a virus-like particle.

38. The nucleic acid of claim 24 provided in a vector or a recombinant host cell.

39. A polypeptide having the structure of formula (I)
A-L1-B-L2-C (I)
wherein
A is an N-terminal amino acid stretch of an adenovirus penton base protein;
B is an amino acid stretch of an adenovirus penton base protein;
C is a C-terminal amino acid stretch of an adenovirus penton base;
wherein amino acid stretch A comprises beta sheets 1, 2 and 3 of the jellyroll fold domain of said adenovirus;
wherein amino acid stretch A has the following consensus sequence (SEQ ID NO: 21):
(U)1-47PTX1GRNSIRYSX2X3x4PX5X6DTTX7X3YLVDNKSADIASL
NYQNDHSNFX5TTVX9QNNDX10X11PX12EAX13TQTINX14DX15RSR
WGX16X17LKTIX18X19TZ1Z2Z3Z4Z5Z6Z7Z8Z9Z10Z11Z12Z13
Z14Z15
and amino acid stretch A ends on the C-terminal side before Z1 at residue T or at an amino acid from Z1 to Z15, and
U is any or no amino acid
X1 is E or G
X2 is E or S
X3 is L or V
X4 is A or S
X5 is L or Q
X6 is Y or E
X7 is R or K
X8 is V or L
X9 is V or I
X10 is F or Y
X11 is T or S
X12 is A or T or I or G
X13 is S or G
X14 is F or L
X15 is E or D
X16 is A or G
X17 is D or Q
X18 is L or M
X19 is H or R
Z1, if present, is N
Z2, if present, is M
Z3, if present, is P
Z4, if present, is N
Z5, if present, is V or I
Z6, if present, is N
Z7, if present, is E or D
Z8, if present, is Y or F
Z9, if present, is M
Z10, if present, is F or S or Y
Z11, if present, is T or S
Z12, if present, is S or N
Z13, if present, is K
Z14, if present, is F
Z16, if present, is K
wherein amino acid stretch B comprises beta sheets 4 and 5 of the jellyroll fold domain of said adenovirus;
wherein amino acid stretch B of above general formula (I) has the following sequence (SEQ ID NO: 22):
Z17Z18Z19Z20Z21Z22Z23Z24Z25Z26Z27QVYWSLPDX20MX21DP
VTFRSTX22QX23X24NX25PVVGX26ELZ28Z29Z30
and amino acid stretch B begins on the N-terminal side at an amino acid from Z17 to Z27 or at amino acid Q after Z27 and ends on the C-terminal side before Z28 at amino acid L or at an amino acid from Z28 to Z30; and
Z17, if present, is L or S
Z18, if present, is T or P or C
Z19, if present, is T or P
Z20, if present, is P or S or A or R
Z21, if present, is N or D
Z22, if present, is G or V
Z23, if present, is H or T
Z24, if present, is C
Z25, if present, is G
Z26, if present, is A or V or S
Z27, if present, is E or Q
X20 is L or M
X21 is Q or K
X22 is Q or R or S
X23 is V or I
X24 is S or N
X25 is Y or F
X26 is A or V
Z28, if present, is M or L
Z29, if present, is P
Z30, if present, is V or F
wherein amino acid stretch C comprises beta sheets 6, 7 and 8 of the jellyroll fold domain of adenovirus;
wherein amino acid stretch C of above general formula (I) has the following sequence (SEQ ID NO: 23):
Z31Z32Z33ALTDHGTLPLRSSIX27GVQRVTX28TDARRRTCPYVYKA
LGIVX30PX31VLSSRTF
and amino acid stretch C begins on the N-terminal side at an amino acid from Z31 to Z33 or at amino acid A after Z33; and
Z31, if present, is N
Z32, if present, is V
Z33, if present, is P
X27 is R or S or G
X28 is V or I
X29 is Y or H
X30 is A or S
X31 is R or K
wherein L1 and L2 are independently from one another selected from the group consisting of an oligopeptide, a polypeptide, a protein, and a protein complex; and
wherein said oligopeptide, polypeptide, protein and protein complex, respectively, are either essentially non-adenoviral or, if adenoviral, are from an adenovirus having a different serotype compared to the serotype of the adenovirus from which said amino acid stretches A, B and C are derived.

40. The polypeptide of claim 39, wherein amino acid stretch A is selected from the group consisting of the following sequences:
from an amino acid selected from positions 1 to 48, to an amino acid selected from positions 129 to 144 of UniProt Acc. No. Q2Y0H9;
from an amino acid selected from positions 1 to 48, to an amino acid selected from positions 129 to 144 of UniProt Acc. No. P03276;
from an amino acid selected from positions 1 to 44, to an amino acid selected from positions 125 to 140 of UniProt Acc. No. Q2KSF3;
from an amino acid selected from positions 1 to 48, to an amino acid selected from positions 129 to 144 of UniProt Acc. No. P12538;
from an amino acid selected from positions 1 to 48, to an amino acid selected from positions 129 to 144 of UniProt Acc. No. Q9JFT6;
from an amino acid selected from positions 1 to 48, to an amino acid selected from positions 129 to 144 of UniProt Acc. No. D2DM93;
from an amino acid selected from positions 1 to 38, to an amino acid selected from positions 119 to 134 of UniProt Acc. No. P36716;
from an amino acid selected from positions 1 to 35, to an amino acid selected from positions 116 to 131 of UniProt Acc. No. F1DT65;
from an amino acid selected from positions 1 to 43, to an amino acid selected from positions 124 to 139 of UniProt Acc. No. M0QUK0;
from an amino acid selected from positions 1 to 49, to an amino acid selected from positions 130 to 145 of UniProt Acc. No. Q7T941;
from an amino acid selected from positions 1 to 35, to an amino acid selected from positions 116 to 131 of UniProt Acc. No. Q912J1;
from an amino acid selected from positions 1 to 46, to an amino acid selected from positions 127 to 142 of UniProt Acc. No. F8WQN4;
from an amino acid selected from positions 1 to 49, to an amino acid selected from positions 130 to 145 of UniProt Acc. No. E5L3Q9;
from an amino acid selected from positions 1 to 44, to an amino acid selected from positions 125 to 140 of UniProt Acc. No. G9G849;
from an amino acid selected from positions 1 to 46, to an amino acid selected from positions 127 to 142 of UniProt Acc. No. H8PFZ9;
from an amino acid selected from positions 1 to 45, to an amino acid selected from positions 126 to 141 of UniProt Acc. No. F6KSU4;
from an amino acid selected from positions 1 to 46, to an amino acid selected from positions 127 to 142 of UniProt Acc. No. F2WTK5;
from an amino acid selected from positions 1 to 43, to an amino acid selected from positions 124 to 139 of UniProt Acc. No. A0A0A1EWW1;
from an amino acid selected from positions 1 to 43, to an amino acid selected from positions 124 to 139 of UniProt Acc. No. A0A0A1EWX7; and
from an amino acid selected from positions 1 to 44, to an amino acid selected from positions 125 to 140 of UniProt Acc. No. A0A0A1EWZ7.

41. The polypeptide of claim 39, wherein amino acid stretch B is selected from the group consisting of the following sequences:
from an amino acid selected from positions 398 to 409, to an amino acid selected from positions 440 to 443 of UniProt Acc. No. Q2Y0H9;
from an amino acid selected from positions 425 to 436, to an amino acid selected from positions 467 to 470 of UniProt Acc. No. P03276;
from an amino acid selected from positions 379 to 390, to an amino acid selected from positions 421 to 444 of UniProt Acc. No. Q2KSF3;
from an amino acid selected from positions 425 to 436, to an amino acid selected from positions 467 to 470 of UniProt Acc. No. P12538;
from an amino acid selected from positions 398 to 409, to an amino acid selected from positions 440 to 443 of UniProt Acc. No. Q9JFT6;
from an amino acid selected from positions 415 to 426, to an amino acid selected from positions 457 to 460 of UniProt Acc. No. D2DM93;
from an amino acid selected from positions 351 to 362, to an amino acid selected from positions 393 to 397 of UniProt Acc. No. P36716;
from an amino acid selected from positions 370 to 381, to an amino acid selected from positions 413 to 416 of UniProt Acc. No. F1DT65;
from an amino acid selected from positions 388 to 399, to an amino acid selected from positions 440 to 443 of UniProt Acc. No. M0QUK0;
from an amino acid selected from positions 445 to 456, to an amino acid selected from positions 497 to 500 of UniProt Acc. No. Q7T941;
from an amino acid selected from positions 372 to 383, to an amino acid selected from positions 414 to 417 of UniProt Acc. No. Q912J1;
from an amino acid selected from positions 362 to 373, to an amino acid selected from positions 404 to 407 of [Please insert UniProt Acc. No. F8WQN4;
from an amino acid selected from positions 416 to 427, to an amino acid selected from positions 458 to 461 of UniProt Acc. No. E5L3Q9;
from an amino acid selected from positions 372 to 383, to an amino acid selected from positions 420 to 423 of UniProt Acc. No. G9G849;
from an amino acid selected from positions 353 to 364, to an amino acid selected from positions 395 to 398 of UniProt Acc. No. H8PFZ9;
from an amino acid selected from positions 358 to 369, to an amino acid selected from positions 400 to 403 of UniProt Acc. No. F6KSU4;
from an amino acid selected from positions 356 to 367, to an amino acid selected from positions 398 to 401 of UniProt Acc. No. F2WTK5;
from an amino acid selected from positions 352 to 363, to an amino acid selected from positions 394 to 397 of UniProt Acc. No. A0A0A1EWW1;
from an amino acid selected from positions 350 to 361, to an amino acid selected from positions 392 to 395 of UniProt Acc. No. A0A0A1EWX7; and
from an amino acid selected from positions 351 to 362, to an amino acid selected from positions 393 to 396 of UniProt Acc. No. A0A0A1EWZ7.

42. The polypeptide of claim 39, wherein the amino acid stretch C is selected from the group consisting of the following sequences:
from an amino acid selected from positions 492 to 495, to the C-terminal amino acid of UniProt Acc. No. Q2Y0H9;
from an amino acid selected from positions 519 to 522, to the C-terminal amino acid of UniProt Acc. No. P03276;
from an amino acid selected from positions 466 to 469, to the C-terminal amino acid of UniProt Acc. No. Q2KSF3;
from an amino acid selected from positions 492 to 495, to the C-terminal amino acid of UniProt Acc. No. P12538;
from an amino acid selected from positions 465 to 468, to the C-terminal amino acid of UniProt Acc. No. Q9JFT6;
from an amino acid selected from positions 482 to 485, to the C-terminal amino acid of UniProt Acc. No. D2DM93;
from an amino acid selected from positions 419 to 422, to the C-terminal amino acid of UniProt Acc. No. P36716;
from an amino acid selected from positions 438 to 441, to the C-terminal amino acid of [Please insert UniProt Acc. No. F1DT65;
from an amino acid selected from positions 455 to 458, to the C-terminal amino acid of UniProt Acc. No. M0QUK0;
from an amino acid selected from positions 522 to 525, to the C-terminal amino acid of UniProt Acc. No. Q7T941;
from an amino acid selected from positions 439 to 442, to the C-terminal amino acid of UniProt Acc. No. Q912J1;
from an amino acid selected from positions 429 to 432, to the C-terminal amino acid of UniProt Acc. No. F8WQN4;
from an amino acid selected from positions 483 to 486, to the C-terminal amino acid of UniProt Acc. No. E5L3Q9;
from an amino acid selected from positions 445 to 448, to the C-terminal amino acid of UniProt Acc. No. G9G849;
from an amino acid selected from positions 420 to 423, to the C-terminal amino acid of UniProt Acc. No. H8PFZ9;
from an amino acid selected from positions 425 to 428, to the C-terminal amino acid of UniProt Acc. No. F6KSU4;
from an amino acid selected from positions 423 to 426, to the C-terminal amino acid of UniProt Acc. No. F2WTK5;
from an amino acid selected from positions 419 to 422, to the C-terminal amino acid of UniProt Acc. No. A0A0A1EWW1;
from an amino acid selected from positions 417 to 420, to the C-terminal amino acid of UniProt Acc. No. A0A0A1EWX7; and
from an amino acid selected from positions 418 to 421, to the C-terminal amino acid of UniProt Acc. No. A0A0A1EWZ7.

43. The polypeptide according to claim 39 wherein amino acid stretch A has the sequence of positions 1 to 132 of UniProt Acc. No. Q2Y0H9, amino acid stretch B has the sequence of positions 407 to 442 of UniProt Acc. No. Q2Y0H9, and amino acid stretch C has the sequence of positions 493 to 544 of UniProt Acc. No. Q2Y0H9.

44. The polypeptide according to claim 39, wherein L1 and/or L2 is an oligopeptide having a sequence of 4 to 40 amino acids being selected from amino acids G and S.

45. The polypeptide of claim 44 wherein L1 and/or L2 is independently selected from the group consisting of GGGS (SEQ ID NO: 24) and GGSGGS (SEQ ID NO: 25).

46. The polypeptide according to claim 39 wherein one or more of L1 and L2 comprise an antigen.

47. The polypeptide according to any claim 39 wherein one or more of a nucleic acid, a drug, label or a binding partner of a biological binding pair are coupled to one or more of L1 and L2.


SEQUENCE LISTING (29 sequences)

SEQ ID NO: 1 - protein, 544 aa, Human adenovirus C serotype 3
MRRRAVLGGAVVYPEGPPPSYESVMQQQAAMIQPPLEAPFVPPRYLAPTEGRNSIRYSELSPLYDTTKLYLVDNKSADIASLNYQNDHSNFLTTVVQNNDFTPTEASTQTINFDERSRWGGQLKTIMHTNMPNVNEYMFSNKFKARVMVSRKAPEGVTVNDTYDHKEDILKYEWFEFILPEGNFSATMTIDLMNNAIIDNYLEIGRQNGVLESDIGVKFDTRNFRLGWDPETKLIMPGVYTYEAFHPDIVLLPGCGVDFTESRLSNLLGIRKRHPFQEGFKIMYEDLEGGNIPALLDVTAYEESKKDTTTETTTLAVAEETSEDDDITRGDTYITEKQKREAAAAEVKKELKIQPLEKDSKSRSYNVLEDKINTAYRSWYLSYNYGNPEKGIRSWTLLTTSDVTCGAEQVYWSLPDMMQDPVTFRSTRQVNNYPVVGAELMPVFSKSFYNEQAVYSQQLRQATSLTHVFNRFPENQILIRPPAPTITTVSENVPALTDHGTLPLRSSIRGVQRVTVTDARRRTCPYVYKALGIVAPRVLSSRTF

SEQ ID NO: 2 - protein, 571 aa, Human adenovirus C serotype 2
MQRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRSLTKDKQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGDGAGGGNNSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTSQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 3 - protein, 535 aa, Human adenovirus E serotype 4
MMRRAYPEGPPPSYESVMQQAMAAAAAIQPPLEAPYVPPRYLAPTEGRNSIRYSELTPLYDTTRLYLVDNKSADIASLNYQNDHSNFLTTVVQNNDFTPTEASTQTINFDERSRWGGQLKTIMHTNMPNVNQFMYSNKFKARVMVSRKTPNGVTVGDNYDGSQDELKYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQIMYEDLDGGNIPALLDVEAYEKSKEESVAAATTAVATASTEVRDDNFASAAAVAAVKADETKSKIVIQPVEKDSKERSYNVLSDKKNTAYRSWYLAYNYGDRDKGVRSWTLLTTSDVTCGVEQVYWSLPDMMQDPVTFRSTHQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGIVAPRVLSSRTF

SEQ ID NO: 4 - protein, 571 aa, Human adenovirus C serotype 5
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 5 - protein, 544 aa, Human adenovirus B serotype 7
MRRRAVLGGAMVYPEGPPPSYESVMQQQAAMIQPPLEAPFVPPRYLAPTEGRNSIRYSELSPLYDTTKLYLVDNKSADIASLNYQNDHSNFLTTVVQNNDFTPTEASTQTINFDERSRWGGQLKTIMHTNMPNVNEYMFSNKFKARVMVSRKAPEGVIVNDTYDHKEDILKYEWFEFTLPEGNFSATMTIDLMNNAIIDNYLEIGRQNGVLESDIGVKFDTRNFRLGWDPETKLIMPGVYTYEAFHPDIVLLPGCGVDFTESRLSNLLGIRKRHPFQEGFKIMYEDLEGGNIPALLDVTAYEESKKDTTTETTTLAVAEETSEDDNITRGDTYITEKQKREAAAAEVKKELKIQPLEKDSKSRSYNVLEDKINTAYRSWYLSYNYGNPEKGIRSWTLLTTSDVTCGAEQVYWSLPDMMQDPVTFRSTRQVNNYPVVGAELMPVFSKSFYNEQAVYSQQLRQATSLTHVFNRFPENQILIRPPAPTITTVSENVPALTDHGTLPLRSSIRGVQRVTVTDARRRTCPYVYKALGIVAPRVLSSRTF

SEQ ID NO: 6 - protein, 557 aa, Human adenovirus B serotype 11
MRRVVLGGAVVYPEGPPPSYESVMQQQATAVMQSPLEAPFVPPRYLAPTEGRNSIRYSELAPQYDTTRLYLVDNKSADIASLNYQNDHSNFLTTVVQNNDFTPTEASTQTINFDERSRWGGQLKTIMHTNMPNVNEYMFSNNFKARVMVSRKPPEGAAVGDTYDHKQDILEYEWFEFTLPEGNFSVTMTIDLMNNAIIDNYLKVGRQNGVLESDIGVKFDTRNFKLGWDPETKLIMPGVYTYEAFHPDIVLLPGCGVDFTESRLSNLLGIRKKQPFQEGFKILYEDLEGGNIPALLDVDAYENSKKEQKAKIEAAAEAKANIVASDSTRVANAGEVRGDNFAPTPVPTAESLLADVSGGTDVKLTIQPVEKDSKNRSYNVLEDKINTAYRSWYLSYNYGDPEKGVRSWTLLTTSDVTCGAEQVYWSLPDMMQDPVTFRSTRQVSNYPVVGAELMPVFSKSFYNEQAVYSQQLRQSTSLTHVFNRFPENQILIRPPAPTITTVSENVPALTDHGTLPLRSSIRGVQRVTVTDARRRTCPYVYKALGIVAPRVLSSRTF

SEQ ID NO: 7 - protein, 497 aa, Human adenovirus A serotype 12
MRRAVELQTVAFPETPPPSYETVMAAAPPYVPPRYLGPTEGRNSIRYSELSPLYDTTRVYLVDNKSSDIASLNYQNDHSNFLTTVVQNNDYSPIEAGTQTINFDERSRWGGDLKTILHTNMPNVNDFMFTTKFKARVMVARKTNNEGQTILEYEWAEFVLPEGNYSETMTIDLMNNAIIEHYLRVGRQHGVLESDIGVKFDTRNFRLGWDPETQLVTPGVYTNEAFHPDIVLLPGCGVDFTESRLSNILGIRKRQPFQEGFVIMYEHLEGGNIPALLDVKKYENSLQDQNTVRGDNFIALNKAARIEPVETDPKGRSYNLLPDKKNTKYRSWYLAYNYGDPEKGVRSWTLLTTPDVTGGSEQVYWSLPDMMQDPVTFRSSRQVSNYPVVAAELLPVHAKSFYNEQAVYSQLIRQSTALTRVFNRFPENQILVRPPAATITTVSENVPALTDHGTLPLRSSISGVQRVTITDARRRTCPYVYKALGIVSPRVLSSRTF

SEQ ID NO: 8 - protein, 517 aa, Human adenovirus D serotype 17
MRRAVVSSSPPPSYESVMAQATLEVPFVPPRYMAPTEGRNSIRYSELAPLYDTTRVYLVDNKSADIASLNYQNDHSNFLTTVVQNNDFTPAEASTQTINFDERSRWGGDLKTILHTNMPNVNEYMFTSKFKARVMVARKHPQGVEATDLSKDILEYEWFEFTLPEGNFSETMTIDLMNNAILENYLQVGRQNGVLESDIGVKFDSRNFKLGWDPVTKLVMPGVYTYEAFHPDVVLLPGCGVDFTESRLSNLLGIRKKQPFQEGFRIMYEDLEGGNIPALLDVPKYLESKKKLEEALENAAKANGPARGDSSVSREVEKAAEKELVIEPIKQDDSKRSYNLIEGTMDTLYRSWYLSYTYGDPEKGVQSWTLLTTPDVTCGAEQVYWSLPDLMQDPVTFRSTQQVSNYPVVGAELMPFRAKSFYNDLAVYSQLIRSYTSLTHVFNRFPDNQILCRPPAPTITTVSENVPALTDHGTLPLRSSIRGVQRVTVTDARRRTCPYVYKALGIVAPRVLSSRTF

SEQ ID NO: 9 - protein, 520 aa, Human adenovirus 25
MRRAVVSSSPPPSYESVMAQATLEVPFVPPRYMAPTEGRNSIRYSELAPQYDTTRVYLVDNKSADIASLNYQNDHSNFLTTVVQNNDFTPAEASTQTINFDERSRWGGDLKTILHTNMPNVNEYMFTSKFKARVMVARKHPENVDKTDLSQDKLEYEWFEFTLPEGNFSETMTIDLMNNAILENYLQVGRQNGVLESDIGVKFDSRNFKLGWDPVTKLVMPGVYTYEAFHPDVVLLPGCGVDFTESRLSNLLGIRKKQPFQEGFRIMYEDLEGGNIPALLDTKKYLDSKKELEDAAKEAAKQQGDGAVTRGDTHLTVAQEKAAEKELVIVPIEKDESNRSYNLIKDTHDTMYRSWYLSYTYGDPEKGVQSWTLLTTPDVTCGAEQVYWSLPDLMQDPVTFRSTQQVSNYPVVGAELMPFRAKSFYNDLAVYSQLIRSYTSLTHVFNRFPDNQILCRPPAPTITTVSENVPALTDHGTLPLRSSIRGVQRVTVTDARRRTCPYVYKALGIVAPRVLSSRTF

SEQ ID NO: 10 - protein, 561 aa, Human adenovirus B serotype 35
MRRVVLGGAVVYPEGPPPSYESVMQQQQATAVMQSPLEAPFVPPRYLAPTEGRNSIRYSELAPQYDTTRLYLVDNKSADIASLNYQNDHSNFLTTVVQNNDFTPTEASTQTINFDERSRWGGQLKTIMHTNMPNVNEYMFSNKFKARVMVSRKPPDGAAVGDTYDHKQDILEYEWFEFTLPEGNFSVTMTIDLMNNAIIDNYLKVGRQNGVLESDIGVKFDTRNFKLGWDPETKLIMPGVYTYEAFHPDIVLLPGCGVDFTESRLSNLLGIRKKQPFQEGFKILYEDLEGGNIPALLDVDAYENSKKEQKAKIEAATAAAEAKANIVASDSTRVANAGEVRGDNFAPTPVPTAESLLADVSEGTDVKLTIQPVEKDSKNRSYNVLEDKINTAYRSWYLSYNYGDPEKGVRSWTLLTTSDVTCGAEQVYWSLPDMMKDPVTFRSTRQVSNYPVVGAELMPVFSKSFYNEQAVYSQQLRQSTSLTHVFNRFPENQILIRPPAPTITTVSENVPALTDHGTLPLRSSIRGVQRVTVTDARRRTCPYVYKALGIVAPRVLSSRTF

SEQ ID NO: 11 - protein, 519 aa, Human adenovirus D37
MRRAVVSSSPPPSYESVMAQATLEVPFVPPRYMAPTEGRNSIRYSELAPLYDTTRVYLVDNKSADIASLNYQNDHSNFLTTVVQNNDFTPAEASTQTINFDERSRWGGDLKTILHTNMPNVNEYMFTSKFKARVMVARKKAEGADANDRSKDILEYQWFEFTLPEGNFSETMTIDLMNNAILENYLQVGRQNGVLESDIGVKFDSRNFKLGWDPVTKLVMPGVYTYEAFHPDVVLLPGCGVDFTESRLSNLLGIRKKQPFQEGFRIMYEDLVGGNIPALLNVKEYLKDKEEAGKADANTIKAQNDAVPRGDNYASAAEAKAAGKEIELKAILKDDSDRSYNVIEGTTDTLYRSWYLSYTYGDPEKGVQSWTLLTTPDVTCGAEQVYWSLPDLMQDPVTFRSTQQVSNYPVVGAELMPFRAKSFYNDLAVYSQLIRSYTSLTHVFNRFPDNQILCRPPAPTITTVSENVPALTDHGTLPLRSSIRGVQRVTVTDARRRTCPYVYKALGIVAPRVLSSRTF

SEQ ID NO: 12 - protein, 508 aa, Human adenovirus F serotype 41
MRRAVGVPPVMAYAEGPPPSYESVMGSADSPATLEALYVPPRYLGPTEGRNSIRYSELAPLYDTTRVYLVDNKSADIASLNYQNDHSNFQTTVVQNNDFTPAEAGTQTINFDERSRWGADLKTILRTNMPNINEFMSTNKFKARLMVEKKNKETGLPRYEWFEFTLPEGNYSETMTIDLMNNAIVDNYLEVGRQNGVLESDIGVKFDTRNFRLGWDPVTKLVMPGVYTNEAFHPDIVLLPGCGVDFTQSRLSNLLGIRKRLPFQEGFQIMYEDLEGGNIPALLDVTKYEASIQKAKEEGKEIGDDTFATRPQDLVIEPVAKDSKNRSYNLLPNDQNNTAYRSWFLAYNYGDPNKGVQSWTLLTTADVTCGSQQVYWSLPDMMQDPVTFRPSTQVSNYPVVGVELLPVHAKSFYNEQAVYSQLIRQSTALTHVFNRFPENQILVRPPAPTITTVSENVPALTDHGTLPLRSSISGVQRVTITDARRRTCPYVHKALGIVAPKVLSSRTF

SEQ ID NO: 13 - protein, 575 aa, Gorilla gorilla adenovirus B7
MMRRAVLGGAVVYPEGPPPSYESVMQQQAAAVMQPSLEAPFVPPRYLAPTEGRNSIRYSELAPQYDTTRLYLVDNKSADIASLNYQNDHSNFLTTVVQNNDFTPTEASTQTINFDERSRWGGQLKTIMHTNMPNVNEYMFSNKFKARVMVSREASKIDSEKNDRSKDTLKYEWFEFTLPEGNFSATMTIDLMNNAIIDNYLAVGRQNGVLQSDIGVKFDTRNFRLGWDPVTKLVMPGVYTYEAFHPDIVLLPDCGVDFTESRLSNLLGIRKRHPFQEGFKIMYEDLEGGNIPALLDVAEYEKSKKEIASSTTTTAVTTVARNVADTSVEAVAVAVVDTIKAENDSAVRGDNFQSKNDMKASEEVTVVPVSPPTVTETETKEPTIKPLEKDTKDRSYNVISGTNDTAYRSWYLAYNYGDPEKGVRSWTLLTTSDVTCGAEQVYWSLPDMMQDPVTFRSTRQVSNYPVVGAELMPVFSKSFYNEQAVYSQQLRQTTSLTHIFDRFPENQILIRPPAPTITTVSENVPALTDHGTLPLRSSIRGVQRVTVTDARRRTCPYVYKALGIVAPRVLSSRTF

SEQ ID NO: 14 - protein, 532 aa, Chimpanzee adenovirus Y25
MMRRAYPEGPPPSYESVMQQAMAAAAAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFMYSNKFKARVMVSRKTPNGVTVTDGSQDILEYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQIMYEDLEGGNIPALLDVDAYEKSKEESAAAATAAVATASTEVRGDNFASPAAVAAAEAAETESKIVIQPVEKDSKDRSYNVLPDKINTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGIVAPRVLSSRTF

SEQ ID NO: 15 - protein, 508 aa, Simian adenovirus 18
MRRAVGVPPVMAYAEGPPPSYETVMGAADSPATLEALYVPPRYLGPTEGRNSIRYSELAPLYDTTRVYLVDNKSADIASLNYQNDHSNFLTTVVQNNDFTPVEAGTQTINFDERSRWGGDLKTILRTNMPNINEFMSTNKFRARLMVEKVNKETNAPRYEWFEFTLPEGNYSETMTIDLMNNAIVDNYLEVGRQNGVLESDIGVKFDTRNFRLGWDPVTKLVMPGVYTNEAFHPDIVLLPGCGVDFTQSRLSNLLGIRKRMPFQAGFQIMYEDLEGGNIPALLDVAKYEASIQKAREQGQEIRGDNFTVIPRDVEIVPVEKDSKDRSYNLLPGDQTNTAYRSWFLAYNYGDPEKGVRSWTLLTTTDVTCGSQQVYWSLPDMMQDPVTFRPSSQVSNYPVVGVELLPVHAKSFYNEQAVYSQLIRQSTALTHVFNRFPENQILVRPPAPTITTVSENVPALTDHGTLPLRSSISGVQRVTITDARRRTCPYVHKALGIVAPKVLSSRTF

SEQ ID NO: 16 - protein, 512 aa, Simian adenovirus 20
MRRAVAIPSAAVALGPPPSYESVMASANLQAPLENPYVPPRYLEPTGGRNSIRYSELTPLYDTTRLYLVDNKSADIATLNYQNDHSNFLTSVVQNSDYTPAEASTQTINLDDRSRWGGDLKTILHTNMPNVNEFMFTNSFRAKLMVAHETNKDPVYKWVELTLPEGNFSETMTIDLMNNAIVDHYLAVGRQNGVKESEIGVKFDTRNFRLGWDPQTELVMPGVYTNEAFHPDVVLLPGCGVDFTYSRLSNLLGIRKRMPFQEGFQIMYEDLVGGNIPALLDVPAYEASITTVAAKEVRGDNFEAAAAAAATGAQPQAAPVVRPVTQDSKGRSYNIITGTNNTAYRSWYLAYNYGDPEKGVRSWTLLTTPDVTCGSEQVYWSMPDMYVDPVTFRSSQQVSSYPVVGAELLPIHSKSFYNEQAVYSQLIRQQTALTHVFNRFPENQILVRPPAPTITTVSENVPALTDHGTLPLQNSIRGVQRVTITDARRRTCPYVYKALGIVAPRVLSSRTF

SEQ ID NO: 17 - protein, 511 aa, Simian adenovirus 49
MRRAVPAAAIPATVAYADPPPSYESVMAGVPATLEAPYVPPRYLGPTEGRNSIRYSELAPLYDTTRVYLVDNKSADIASLNYQNDHSNFLTTVVQNNDFTPVEAGTQTINFDERSRWGGQLKTILHTNMPNVNEFMFTNSFRAKVMVSRKQNEEGQTELEYEWVEFVLPEGNYSETMTLDLMNNAIVDHYLLVGRQNGVLESDIGVKFDTRNFRLGWDPVTKLVMPGVYTNEAFHPDVVLLPGCGVDFTQSRLSNLLGIRKRQPFQEGFRIMYEDLEGGNIPALLNVKAYEDSIAAAMRKHNLPLRGDVFAVQPQEIVIQPVEKDGKERSYNLLPDDKNNTAYRSWYLAYNYGDPLKGVRSWTLLTTPDVTCGSEQVYWSLPDLMQDPVTFRPSSQVSNYPVVGAELLPLQAKSFYNEQAVYSQLIRQSTALTHVFNRFPENQILVRPPAATITTVSENVPALTDHGTLPLRSSISGVQRVTITDARRRTCPYVYKALGIVAPRVLSSRTF

SEQ ID NO: 18 - protein, 505 aa, Rhesus adenovirus 51
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRAKLMVEKSNAETRQPRYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPALLDVSKYEASIQRAKAEGREIRGDTFAVAPQDLEIVPLTKDSKDRSYNIINNTTDTLYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 19 - protein, 503 aa, Rhesus adenovirus 52
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVKKVENQPPEYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPALLDVTKYEQSVQRAKAEGREIRGDTFAVSPQDLVIEPLEHDSKNRSYNLLPNKTDTAYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 20 - protein, 504 aa, Rhesus adenovirus 53
MRRAVRVTPAVYAEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVEKTSGQPPKYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPGLLDVPAYEQSLQQAQEEGRVTRGDTFATAPNEVVIKPLLKDSKDRSYNIITDTTDTLYRSWFLAYNYGDPENGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 21 - protein, 144 aa, Artificial Sequence
  Consensus sequence for amino acid stretch A
  MISC_FEATURE (1)..(47): No amino acid present in this position or any amino acid
  MISC_FEATURE (50)..(50): E or G
  MISC_FEATURE (59)..(59): E or S
  MISC_FEATURE (60)..(60): L or V
  MISC_FEATURE (61)..(61): A or S
  MISC_FEATURE (63)..(63): L or Q
  MISC_FEATURE (64)..(64): Y or E
  MISC_FEATURE (68)..(68): R or K
  MISC_FEATURE (69)..(69): V or L
  MISC_FEATURE (92)..(92): L or Q
  MISC_FEATURE (96)..(96): V or I
  MISC_FEATURE (101)..(101): F or Y
  MISC_FEATURE (102)..(102): T or S
  MISC_FEATURE (104)..(104): A or T ot I or G
  MISC_FEATURE (107)..(107): S or G
  MISC_FEATURE (113)..(113): F or L
  MISC_FEATURE (115)..(115): E or D
  MISC_FEATURE (121)..(121): A or G
  MISC_FEATURE (122)..(122): D or Q
  MISC_FEATURE (127)..(127): L or M
  MISC_FEATURE (128)..(128): H or R
  MISC_FEATURE (130)..(130): no amino acid or, if present, N
  MISC_FEATURE (131)..(131): no amino acid or, if present, M
  MISC_FEATURE (132)..(132): no amino acid or, if present, P
  MISC_FEATURE (133)..(133): no amino acid or, if present, N
  MISC_FEATURE (134)..(134): no amino acid or, if present, V or I
  MISC_FEATURE (135)..(135): no amino acid or, if present, N
  MISC_FEATURE (136)..(136): no amino acid or, if present, E or D
  MISC_FEATURE (137)..(137): no amino acid or, if present, Y or F
  MISC_FEATURE (138)..(138): no amino acid or, if present, M
  MISC_FEATURE (139)..(139): no amino acid or, if present, F or S or Y
  MISC_FEATURE (140)..(140): no amino acid or, if present, T or S
  MISC_FEATURE (141)..(141): no amino acid or, if present, S or N
  MISC_FEATURE (142)..(142): no amino acid or, if present, K
  MISC_FEATURE (143)..(143): no amino acid or, if present, F
  MISC_FEATURE (144)..(144): no amino acid or, if present, K
XXXXXXXXXXXXXXXXXXXXXXXXXXXXXXXXXXXXXXXXXXXXXXXPTXGRNSIRYSXXXPXXDTTXXYLVDNKSADIASLNYQNDHSNFXTTVXQNNDXXPXEAXTQTINXDXRSRWGXXLKTIXXTXXXXXXXXXXXXXXX

SEQ ID NO: 22 - protein, 46 aa, Artificial Sequence
  Consensus sequence for amino acid stretch B
  MISC_FEATURE (1)..(1): no amino acid or, if present, L or S
  MISC_FEATURE (2)..(2): no amino acid or, if present, T or P or C
  MISC_FEATURE (3)..(3): no amino acid or, if present, T or P
  MISC_FEATURE (4)..(4): no amino acid or, if present, P or S or A or R
  MISC_FEATURE (5)..(5): no amino acid or, if present, N or D
  MISC_FEATURE (6)..(6): no amino acid or, if present, G or V
  MISC_FEATURE (7)..(7): no amino acid or, if present, H or T
  MISC_FEATURE (8)..(8): no amino acid or, if present, C
  MISC_FEATURE (9)..(9): no amino acid or, if present, G
  MISC_FEATURE (10)..(10): no amino acid or, if present,  A or V or S
  MISC_FEATURE (11)..(11): no amino acid or, if present,  E or Q
  MISC_FEATURE (20)..(20): L or M
  MISC_FEATURE (22)..(22): Q or K
  MISC_FEATURE (31)..(31): Q or R or S
  MISC_FEATURE (33)..(33): V or I
  MISC_FEATURE (34)..(34): S or N
  MISC_FEATURE (36)..(36): Y or F
  MISC_FEATURE (41)..(41): A or V
  MISC_FEATURE (44)..(44): no amino acid or, if present, M or L
  MISC_FEATURE (45)..(45): no amino acid or, if present, P
  MISC_FEATURE (46)..(46): no amino acid or, if present, V or F
XXXXXXXXXXXQVYWSLPDXMXDPVTFRSTXQXXNXPVVGXELXXX

SEQ ID NO: 23 - protein, 53 aa, Artificial Sequence
  Consensus sequence for amino acid stretch C
  MISC_FEATURE (1)..(1): no amino acid or, if present, N
  MISC_FEATURE (2)..(2): no amino acid or, if present, V
  MISC_FEATURE (3)..(3): no amino acid or, if present, P
  MISC_FEATURE (18)..(18): R or S or G
  MISC_FEATURE (25)..(25): V or I
  MISC_FEATURE (44)..(44): A or S
  MISC_FEATURE (46)..(46): R or K
XXXALTDHGTLPLRSSIXGVQRVTXTDARRRTCPYVYKALGIVXPXVLSSRTF

SEQ ID NO: 24 - protein, 4 aa, Artificial Sequence
  Linking segment
GGGS

SEQ ID NO: 25 - protein, 6 aa, Artificial Sequence
  Linking segment
GGSGGS

SEQ ID NO: 26 - DNA, 778 bp, Artificial Sequence
  Construct DNAsegVAJB-CHIK
ggatccatgaggagacgagccgtgctaggcggagcggtggtgtatccggagggtcctcctccttcttacgagagcgtgatgcagcaacaggcggcgatgatacagcccccactggaggctcccttcgtacccccacggtacctggcgcctacggaagggagaaacagcattcgttactcggagctgtcgcccctgtacgataccaccaagttgtatctggtggacaacaagtcggcggacatcgcctccctgaactatcagaacgaccacagcaacttcctgaccacggtggtgcagaacaatgactttacccccacggaggctagcacccagaccatcaactttgacgagcggtcgcgatggggcggtcagctgaagaccatcatgcacaccaacatgcccggaggtgaaaacctgtattttcagagcaccaaagataactttaacgtgtataaagcgacccgcccgtatctggcgcatggaggtgcagagcaggtctactggtcgctccctgacatgatgcaagacccagtcaccttccgctccacaagacaagtcaacaactacccagtggtgggtgcagagcttatgcccggtggaagcggaggtagcgttcctgctctcacagatcacgggaccctgccgttacgcagcagtatccggggagtccagcgcgtgaccgttactgacgccagacgccgcacctgtccctacgtttacaaggccctgggcatagtcgcgccgcgcgttctttcaagccgcactttctgataagctt

SEQ ID NO: 27 - DNA, 3699 bp, Artificial Sequence
  Construct pACEBac_VAJB-CHIK
accggttgacttgggtcaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattgacttgggtcgctcttcctgtggatgcgcagatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatgtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgccataacttcgtatagcatacattatacgaagttatctgtaactataacggtcctaaggtagcgagtttaaacactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatccatgaggagacgagccgtgctaggcggagcggtggtgtatccggagggtcctcctccttcttacgagagcgtgatgcagcaacaggcggcgatgatacagcccccactggaggctcccttcgtacccccacggtacctggcgcctacggaagggagaaacagcattcgttactcggagctgtcgcccctgtacgataccaccaagttgtatctggtggacaacaagtcggcggacatcgcctccctgaactatcagaacgaccacagcaacttcctgaccacggtggtgcagaacaatgactttacccccacggaggctagcacccagaccatcaactttgacgagcggtcgcgatggggcggtcagctgaagaccatcatgcacaccaacatgcccggaggtgaaaacctgtattttcagagcaccaaagataactttaacgtgtataaagcgacccgcccgtatctggcgcatggaggtgcagagcaggtctactggtcgctccctgacatgatgcaagacccagtcaccttccgctccacaagacaagtcaacaactacccagtggtgggtgcagagcttatgcccggtggaagcggaggtagcgttcctgctctcacagatcacgggaccctgccgttacgcagcagtatccggggagtccagcgcgtgaccgttactgacgccagacgccgcacctgtccctacgtttacaaggccctgggcatagtcgcgccgcgcgttctttcaagccgcactttctgataagcttccatcaactttgacgagcggtcgcgatggggcggtcagctgaagaccatcatgcacaccaacatgcccaacgtgaacgagtacatgttcagcaacaagttcaaggcgagggagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctagggtatacccatctaattggaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccaca

SEQ ID NO: 28 - protein, 254 aa, Artificial Sequence
  Protein VAJB-CHIK
MRRRAVLGGAVVYPEGPPPSYESVMQQQAAMIQPPLEAPFVPPRYLAPTEGRNSIRYSELSPLYDTTKLYLVDNKSADIASLNYQNDHSNFLTTVVQNNDFTPTEASTQTINFDERSRWGGQLKTIMHTNMPGGENLYFQSTKDNFNVYKATRPYLAHGGAEQVYWSLPDMMQDPVTFRSTRQVNNYPVVGAELMPGGSGGSVPALTDHGTLPLRSSIRGVQRVTVTDARRRTCPYVYKALGIVAPRVLSSRTF

SEQ ID NO: 29 - protein, 18 aa, Chikungunya virus
STKDNFNVYKATRPYLAH